(19) United States
(12) Patent Application Publication
(10) Pub. No.: US 2024/0049974 A1
(43) Pub. Date: Feb. 15, 2024

(54) SYSTEMS, APPARATUS AND METHODS FOR ACQUISITION, STORAGE AND ANALYSIS OF HEALTH AND ENVIRONMENTAL DATA

(71) Applicant: Circadia Technologies, Ltd., London (GB)

(72) Inventors: Michal Maslik, London (GB); Timo Lauteslager, London (GB); Fares Siddiqui, London (GB)

(21) Appl. No.: 18/271,227

(22) PCT Filed: Jan. 7, 2022
(86) PCT No.: PCT/IB2022/050109

§ 371 Date: Jul. 6, 2023

Related U.S. Application Data
(60) Provisional application No. 63/134,948, filed on Jan. 7, 2021.

(51) Int. Cl. A61B 5/05; A61B 5/11; A61B 5/113; A61B 5/0205; A61B 5/00; G01S 7/41; G01S 13/02; G01S 13/00

(52) U.S. Cl. CPC A61B 5/05; A61B 5/1126; A61B 5/113; A61B 5/0205; A61B 5/4812; A61B 5/0022; A61B 5/742; G01S 7/415; G01S 13/0209; G01S 13/003; A61B 5/024

(57) ABSTRACT

An apparatus and methods for non-contact monitoring of one or more persons/patients are disclosed. The apparatus includes a radar system configured for acquiring motion and proximity data of one or more persons at a plurality of distances, a processor configured for processing the acquired motion and proximity data to identify one or more physiological and/or behavioral features of one or more persons, and a transmitter configured for transmitting the one or more physiological and/or behavioral features of one or more persons to a remote device. In various embodiments, the apparatus may include a wearable sensor, a light or ambient sensor, a microphone and a speaker, or one or more buttons for user input. In various embodiments, a system for monitoring includes a plurality of apparatuses in a mesh network for sharing data from the plurality of apparatuses.

### RELATED APPLICATIONS

This application claims the benefit of U.S. Provisional Patent Application No. 63/134,948 filed Jan. 7, 2021, the content of which is incorporated by this reference its entirety for all purposes as if fully set forth herein.

### TECHNICAL FIELD

The subject matter described herein relates to apparatus and methods for continuous, long-term monitoring of physiological functions and behaviors of one or more persons. More specifically, the disclosed monitoring system is configured for monitoring individual persons or patients in home and clinical settings.

### BACKGROUND

Continuous and long-term monitoring of vital signs and sleep in low acuity settings, such as the general ward, skilled nursing facility, inpatient rehabilitation facility, or home, may be challenging with conventional monitoring standards and technology. The current clinical standard for respiratory rate monitoring in low acuity settings is a manual spot check, typically performed every 4-8 hours by a nurse. Sleep monitoring may not be part of standard clinical practice at all, unless a suspicion exists of a sleep disorder.

Contact-based continuous monitoring technologies exist (e.g., electrodes, wearables, finger probes), but may be uncomfortable. Consequently, patients may be unlikely to adhere to long-term monitoring, unless enforced by healthcare facility staff. Patient monitoring in the home environment may be particularly challenging, as it may involve limited contact between staff and patient, causing low adherence. Existing devices often require trained staff for device setup. In addition, the home environment poses challenges such as patients having a bed partner, which may confound monitor signal quality.

Despite these challenges, there are clear indications that continuous monitoring can be beneficial for early detection of adverse events in various healthcare settings. Accordingly, a need exists for vital sign monitoring systems that address the forgoing and other concerns.

The information included in this Background section of the specification, including any references cited herein and any description or discussion thereof, is included for technical reference purposes only and is not to be regarded as subject matter by which the scope of the disclosure is to be bound.

### SUMMARY

In accordance with various embodiments, an apparatus for non-contact monitoring of a person is provided. The apparatus includes a radar system configured for acquiring motion and proximity data of a person at a plurality of distances; a processor configured for processing the acquired motion and proximity data to identify one or more physiological and/or behavioral features of the person; and a transmitter configured for transmitting the one or more physiological and/or behavioral features of the person to a remote device.

In accordance with various embodiments, a method for non-contact monitoring of a person is provided. The method includes acquiring, via a radar system, motion and proximity data of the person at a plurality of distances; storing, in a memory, the acquired motion and proximity data; processing, via a processor coupled to the memory, the acquired motion and proximity data; identifying, via the processor, one or more physiological and/or behavioral features of the person based on the processed motion and proximity data; and transmitting, via a transmitter, the one or more physiological and/or behavioral features of the person to a remote device.

In accordance with various embodiments, a system for monitoring a plurality of patients is provided. The system includes a plurality of apparatuses in a mesh network comprising at least a first apparatus and a second apparatus, wherein the first apparatus is positioned at a first location within a first local area, wherein the first apparatus comprises: a radar system configured for acquiring motion and proximity data of at least a first patient from the plurality of patients at a plurality of distances; a processor configured for processing the acquired motion and proximity data to identify one or more physiological and/or behavioral features of at least the first patient from the plurality of patients; and a transmitter configured for transmitting the one or more physiological and/or behavioral features of at least the first patient from the plurality of patients to the second apparatus in the mesh network or a remote server.

In accordance with various embodiments, a method for monitoring a plurality of patients is provided. The method includes configuring a plurality of apparatuses in a mesh network comprising at least a first apparatus and a second apparatus, wherein the first apparatus is positioned at a first location within a first local area, wherein at least a first patient from the plurality of patients is present at the first location and being monitored by the first apparatus; acquiring, via a radar system of the first apparatus, motion and proximity data of at least the first patient from the plurality of patients at a plurality of distances; storing, in a memory of the first apparatus, the acquired motion and proximity data; processing, via a processor coupled to the memory, the acquired motion and proximity data; identifying, via the processor, one or more physiological and/or behavioral features of at least the first patient from the plurality of patients based on the processed motion and proximity data; and transmitting, via a transmitter of the first apparatus, the one or more physiological and/or behavioral features of at least the first patient from the plurality of patients to the second apparatus in the mesh network or a remote server.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is a schematic of a monitoring apparatus, in accordance with various embodiments of the present disclosure.

FIG. 2 is a schematic overview of a monitoring system, in accordance with various embodiments of the present disclosure.

FIG. 3A is a perspective view of a monitoring apparatus placed beside a bed, in a home setting, in accordance with various embodiments of the present disclosure.

FIG. 3B is a perspective view of a monitoring apparatus placed beside a bed, in a healthcare setting, in accordance with various embodiments of the present disclosure.

FIG. 4A is a perspective view of a monitoring apparatus, in accordance with various embodiments of the present disclosure.

FIG. 4B is an exploded view of the monitoring apparatus of FIG. 4A, in accordance with various embodiments of the present disclosure.

FIG. 4C is a perspective view illustrating a monitoring apparatus with a magnetic connection to a stand, in accordance with various embodiments of the present disclosure.

FIG. 4D is a perspective view illustrating a magnetic connection of a monitoring apparatus to a wall mount, in accordance with various embodiments of the present disclosure.

FIG. 5 is a schematic view of a plurality of apparatuses in a mesh network, in accordance with various embodiments of the present disclosure.

FIG. 6 is a schematic illustration of a monitoring system showing interconnectivity between two apparatuses with a network server/device, in accordance with various embodiments of the present disclosure.

FIG. 7 is a block diagram illustrating a computer system for use in performing processes and methods provided herein, in accordance with various embodiments.

FIG. 8 is a process flow for a method of non-contact monitoring a person, in accordance with various embodiments of the present disclosure.

FIG. 9 is a process flow for a method of monitoring a plurality of patients, in accordance with various embodiments of the present disclosure.

### DETAILED DESCRIPTION

In accordance with various embodiments of the present disclosure, an apparatus and methods for monitoring of physiological functions and behaviors of one or more persons are described. The disclosed apparatus and methods employ a radar system and one or more sensors to acquire motion and proximity data of one or more persons to process physiological and/or behavior features of the one or more persons. Physiological features include, among others, vital signs, such as but not limited to respiratory rate or heart rate, and respiratory features such as but not limited to respiratory patterns, or duration of inhalation or exhalation, or the likes. Behavior features include, among others, sleep behaviors or movements, for example, such as but not limited to, a bed exit or a fall of the one or more persons being monitored.

In accordance with various embodiments, the monitoring apparatus and system disclosed herein are configured for continuous non-contact monitoring of a person or a patient's vital signs, sleep, behavior, and environmental data, among many others, using multiple sensors including a radar system. In various embodiments, the monitoring apparatus and system can be configured to transmission raw acquired data or process the raw acquired data to produce physiological and/or behavior features of the monitored person/persons/patient/patients. In various embodiments, the raw acquired data or processed features can be transmitted to a remote device, a remote server, or a cloud storage, for health and sleep monitoring and behavioral analysis. As described herein, the monitoring apparatus and system comprise multiple sensors to continuously acquire user motion and proximity data (e.g., raw data), a distance of the user from the apparatus or system, physiological, and behavior data, as well as environmental data in a vicinity of the person/persons/patient/patients being monitored. In various embodiments, the raw acquired data is processed on the monitoring apparatus and system using embedded algorithms. In some embodiments, the raw acquired data and/or processed data (e.g., physiological and behavior data or features) may be stored on the monitoring apparatus and system. In some embodiments, the raw acquired data and/or processed data may also be transmitted for remote storage and processing at a remote device or a remote server. Further details of the disclosed apparatus and system are described with respect to the following figures.

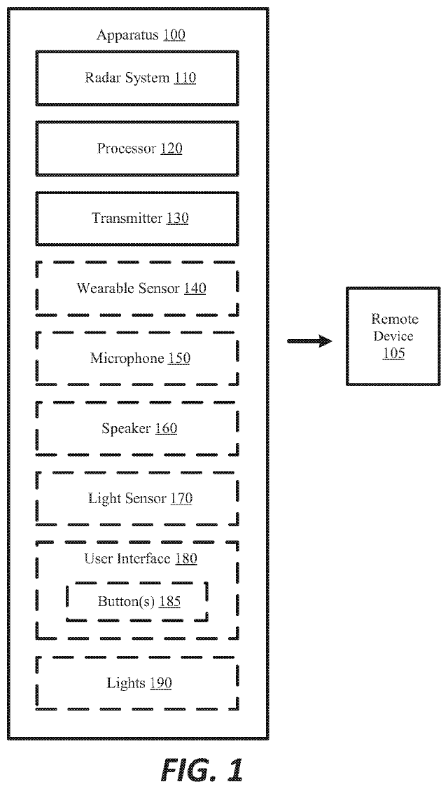

FIG. 1 is a schematic of a monitoring apparatus 100 (or apparatus 100), in accordance with various embodiments. In accordance with the present disclosure, the monitoring apparatus 100 can be configured for non-contact monitoring of a person in a home or school setting, or a patient in a clinical or hospital setting. As disclosed herein, the monitoring apparatus 100 may be configured to determine the presence, distance, and movement of the person from the monitoring apparatus 100. As shown in FIG. 1, the monitoring apparatus 100 includes a radar system 110, a processor 120, and a transmitter 130. In accordance with various embodiments, the monitoring apparatus 100 can include one or more of a wearable sensor 140, a microphone 150, a speaker 160, a light or ambient sensor 170, a user interface 180 having one or more buttons 185, or one or more LED lights 190. In various embodiments, the monitoring apparatus 100 is configured to send data/information to a remote device 105 (a remote server or a storage unit).

In various embodiments, the radar system 110 is configured for acquiring motion and proximity data of a person (or a patient). This acquisition of motion and proximity data can be performed when monitoring the person at a plurality of distances from the monitoring apparatus 100. In various embodiments, the radar system 110 is configured for acquiring the motion and proximity data of the person within a range of distance, e.g., between about 0.01 m and about 30 m, from the monitoring apparatus 100. In various embodiments, the motion and proximity data are acquired within the ranges of distance, e.g., between about 0.05 m and about 20 m, between about 0.1 m and about 10 m, between about 0.2 m and about 5 m, between about 0.3 m and about 5 m, between about 0.3 m and about 3.2 m, between about 0.1 m and about 3.2 m, between about 0.4 m and about 3 m, or between about 0.5 m and about 2.5 m, inclusive of any ranges of distance thereof, from the monitoring apparatus 100. In some embodiments, the radar system 110 can be configured for acquiring the motion and proximity data within a range of distance between about 0.01 m and about 30 m from the monitoring apparatus 100 to determine that the person being monitored is not present in the range of distance.

In various embodiments, the radar system 110 is configured such that the monitoring and acquisition of motion and proximity data occurs at the plurality of distances that are divided into equal-sized bins or bins of different sizes, within the range of distance from the monitoring apparatus 100, e.g., between about 0.01 m and about 30 m, between about 0.05 m and about 20 m, between about 0.1 m and about 10 m, between about 0.2 m and about 5 m, between about 0.3 m and about 5 m, between about 0.3 m and about 3.2 m, between about 0.1 m and about 3.2 m, between about 0.4 m and about 3 m, or between about 0.5 m and about 2.5 m, inclusive of any ranges of distance thereof. In accordance with some embodiments, the number of equal-sized bins or bins of different sizes can range from about 2 to about 1000, from about 5 to about 750, from about 10 to about 500, from about 20 to about 250, from about 25 to about 200, from about 50 to about 100, or any suitable number of bins appropriate for the radar system 110.

In accordance with some embodiments, the radar system 110 of the monitoring apparatus 100 can be configured for location-aware motion sensing modality based on the radiofrequency (RF) signals used in the radar system 110. This modality can provide the distance information of the person being monitored, from the bin at which motion is detected. For example, the radar system 110 can be configured for determining a position of the person or distance of the person from the monitoring apparatus 100 based on the acquired motion and proximity data.

In various embodiments, the radar system 110 includes a transceiver that includes at least one transmitting antenna and at least one receiving antenna. In various embodiments, the radar system 110 includes a single antenna configured for both transmitting and receiving. In various embodiments, the radar system 110 is a monostatic radar system, including one transmitting antenna and one receiving antenna. In some embodiments, the radar system 110 can be a single, monostatic radar system. In accordance with various embodiments, the monostatic radar system includes a single transmitter and receiver pair. A monostatic radar system can be configured to sense motion and distance in 1D space, for example, a linear distance between the radar system and the person/patient being monitored (e.g., as a default configuration).

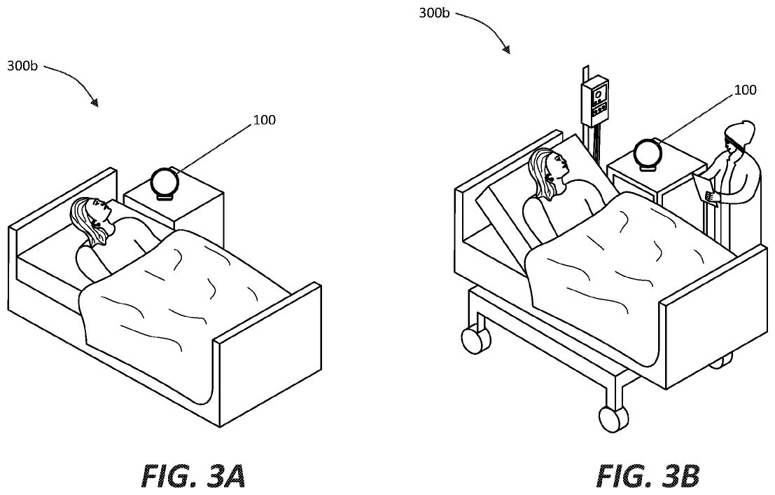

In some embodiments, the radar system 110 can be a pulsed radar architecture, or a stepped-frequency continuous-wave radar (SFCW), or a frequency-modulated continuous-wave radar (FMCW). In various embodiments, the radar system 110 includes a coherent pulsed ultra-wide band (UWB) radar. In various embodiments, the radar system 110 is a multistatic radar system for acquiring motion and proximity data of a plurality of persons. In accordance with various embodiments, the multistatic radar system includes multiple transmitter and receiver antennas. In various embodiments, the multistatic radar system can be configured to sense motion and distance information in 2D or 3D space, for example, a location (including distance from the radar system) of the person/patient being monitored in two- or three-dimensional space. In some embodiments, the multistatic radar system is configured for beamforming to direct a radar beam to one or more specific anatomical portions of various persons of the plurality of persons within the aforementioned ranges of distance from the monitoring apparatus 100. To obtain spatially diverse data in the multistatic radar system, either a single transceiver system may be used in combination with a switch matrix, or multiple transceivers may be used without the need of a switch matrix. In case of multistatic radar data, standard beamforming techniques may be used to adaptively optimize gain in the direction of the person or patient being monitored, and to spatially filter out competing noise sources (e.g., moving objects or persons) in the vicinity of the monitoring apparatus 100. In various embodiments, the multistatic radar system is used along with beamforming, monitoring of multiple persons may be aided by constructing an individual beamformer for each person, according to beamforming theory. By using spatial filtering, multiple persons and patients can be monitored and separating them via data analysis. In various embodiments, the multistatic radar system is used along with beamforming, separate monitoring of a single user's abdomen and thorax may be performed, e.g., when the monitoring apparatus 100 is placed beside a bed of the person or patient, as illustrated in FIGS. 3A and 3B. In accordance with various embodiments, a thoracic and abdominal sensing radar beam may be computed according to beamforming theory. In various embodiments, separate monitoring of abdominal and thoracic respiration-induced displacement may be of relevance for detecting, for example, breathing patterns associated with REM sleep, stress, and paradoxical breathing.

In accordance with various embodiments, the radar system 110 can be configured to employ a transmitted signal having a center frequency of about 1 GHz, about 2 GHz, about 3 GHz, about 3.5 GHz, about 4 GHz, about 4.5 GHz, about 5 GHz, about 5.5 GHz, about 6 GHz, about 6.5 GHz, about 7 GHz, about 7.29 GHz, about 7.5 GHz, about 8 GHz, about 8.5 GHz, about 9 GHz, about 9.5 GHz, about 10 GHz, about 10.5 GHz, about 10.6 GHz, about 11 GHz, about 11.5 GHz, about 12 GHz, about 12.5 GHz, about 13 GHz, about 13.5 GHz, about 14 GHz, about 14.5 GHz, about 15 GHz, inclusive any center frequency between 1 GHz and 15 GHz at 0.01 GHz intervals. Accordingly, the radar system 110 can be configured to operate in an ultra-wideband frequency band ranging from about 3.1 GHz to about 10.6 GHz. In addition, the radar system 110 can operate below the part 15 limit of −41.3 dBm/MHz, in accordance with regulations by the Federal Communications Commission (FCC) for unlicensed transmission of RF signals in the United States, as well as other various regulatory bodies in other parts of the world. In accordance with various embodiments, the aforementioned center frequency provides a high sensitivity to detect respiration-induced chest displacement, for example. In some embodiments, the radar system 110 may be configured to operate in the automotive short-range radar band, e.g., 76 GHz to 81 GHz or in the ISM bands of 24 GHz or 122 GHz.

In various embodiments, the radar system 110 can be configured to detect at a radar frame rate of about 1 frame per second, about 5 frames per second, about 10 frames per second, about 11 frames per second, about 11.5 frames per second, about 12 frames per second, about 12.5 frames per second, about 13 frames per second, about 13.5 frames per second, about 14 frames per second, about 14.5 frames per second, about 15 frames per second, about 15.5 frames per second, about 16 frames per second, 16.2 frames per second, about 16.5 frames per second, about 17 frames per second, about 17.5 frames per second, about 18 frames per second, about 18.5 frames per second, about 19 frames per second, about 19.5 frames per second, about 20 frames per second, about 25 frames per second, about 30 frames per second, about 35 frames per second, about 40 frames per second, about 45 frames per second, about 50 frames per second, about 55 frames per second, about 60 frames per second, about 65 frames per second, about 70 frames per second, about 75 frames per second, about 80 frames per second, about 85 frames per second, about 90 frames per second, about 95 frames per second, about 100 frames per second, about 110 frames per second, about 120 frames per second, about 150 frames per second, inclusive of any frame rates between 1 and 150 frames per second at an interval of 0.1 frame per second. In accordance with various embodiments, the aforementioned frame rates of the radar system 110 are sufficient to accurately acquire motion and proximity data of the person to obtain various physiological and/or behavior features, including for example, respiration and heart rate, of the person or persons being monitored.

In some embodiments, the acquired motion and proximity data include respiratory-induced body movements from thoracic and abdominal areas of the person. In some embodiments, the acquired motion and proximity data include cardiac-induced body movements from thoracic and abdominal areas of the person. Since respiration and heartbeat of the person may cause a displacement in the chest and abdomen, of a few millimeters and sub-millimeter, respectively, the acquired motion and proximity data from these anatomical portions can be used to determine physiological features, such as but not limited to respiration and heart activity. In addition, the monitoring apparatus 100 can be configured to capture heartbeat from pulsatile motions in the limbs (e.g., the cardioballistic effect).

In various embodiments, the acquired motion and proximity data of the person can be processed by the processor 120 for identifying the person from other people present between 0.01 m and 30 m (or any other aforementioned ranges of distance) from the apparatus. From the acquired motion and proximity data, a position of the person from the monitoring apparatus 100 can be determined. In some embodiments, a larger detection range may be set to allow monitoring of a larger living area. The detection range, e.g., the range of distance from the apparatus 110, may be user defined through software, to customize the monitoring apparatus 100 to an individual person or patient's needs. In various embodiments, a radar architecture of the radar system 110 can be configured to provide the ability to sample the entire detection range, e.g., between 0.01 m and 30 m. In various embodiments, the radar system 110 is configured such that a plurality of persons or patients can be monitored simultaneously and separating them by analyzing and identifying the person or patient from other persons or patients via data analysis of the acquired motion or proximity data. In such embodiments, for example, a person and a bed partner (or a patient and another patient in the room) can be monitored in a home setting or multiple beds in a hospital/clinic room in a hospital ward or homecare setting.

In various embodiments, the acquired motion and proximity data includes vital signs of the person. In various embodiments, the acquired motion and proximity data is used to determine a breathing pattern of the person. In various embodiments, the acquired motion and proximity data is used to monitor a heart activity of the person. In various embodiments, the acquired motion and proximity data is used to monitor behavior of the person. In various embodiments, the acquired motion and proximity data includes data captured, for example, but not limited to while the person is asleep, while the person is awake in bed, while the person moves around in a vicinity of the monitoring apparatus 100, when the person moves out of a bed, when the person moves into a bed, or when the person falls down.

As illustrated in FIG. 1, the processor 120 is configured for processing the acquired motion and proximity data to identify one or more physiological and/or behavioral features of the person or persons being monitored, in accordance with various embodiments. In various embodiments, the one or more physiological features include vital signs and/or respiratory features, where the vital signs can be a respiratory rate or a heart rate of the person and where the respiratory features can include various data and features related to respiration, including for example, inhalation and exhalation. In various embodiments, the one or more behavioral features include movement of the person, a distance and location of the person from the monitoring apparatus 100, one of bed occupancy, an activity, such as but not limited to a bed exit, a bed entrance, a fall, or sleep behaviors. In various embodiments, the sleep behaviors of the person may include a pattern of sleep stages that the person goes through during sleep.

In various embodiments, the transmitter 130 of the monitoring apparatus 100 is configured for transmitting the one or more physiological and/or behavioral features of the person to the remote device 105. In some embodiments, the transmitter 130 is configured for transmitting the acquired motion and proximity data of the person (i.e., raw acquired data) to the remote device 105, which may occur prior to processing by the processor 120. In various embodiments, the transmitter 130 is a wired communication component configured to work over Ethernet or USB protocol. In various embodiments, the transmitter 130 is a wireless communication component configured to work over Bluetooth, Wi-Fi, a local area network, a wide area network, or a cellular network.

In various embodiments, the monitoring apparatus 100 may include the wearable sensor 140. In various embodiments, the wearable sensor 140 can include for example, but not limited to a pulse-oximeter, a heart rate monitor, an ultrasound sensor, or a thermometer, or a nearable sensor from a list of an accelerometer, a pressure sensor, optical sensor, including a video-, infrared- or laser-based sensor, a capacitive or touch sensor, or an environmental sensor. In various embodiments, the monitoring apparatus 100 is configured as a hub for collecting sensor data from one or more wearable sensors 140 or another nearable sensor. In such embodiments, the transmitter 120 of the monitoring apparatus 100 can be configured to transmit the collected sensor data from one or more wearable sensors 140 to the remote device 105.

In various embodiments, the monitoring apparatus 100 may include the microphone 150 and the speaker 160. In various embodiments, the microphone 150 and the speaker 160 are configured for communicating with a health care professional or caretaker. In various embodiments, the microphone 150 is used for monitoring a physiological function of the person and the physiological function includes one of respiration, coughing, or snoring. In various embodiments, the microphone 150 can be used for monitoring noise in an environment of the person. In various embodiments, the microphone 150 can be used for monitoring a behavior of the person and the behavior includes one of TV watching, going to bed, or falling down.

In various embodiments, the monitoring apparatus 100 may include the light sensor 170 configured for monitoring a light level of an environment of the person. In various embodiments, the light sensor 170 is used for monitoring a bed time of the person. In various embodiments, the bed time is determined when the light sensor 170 detects that a light in the environment of the person is turned off.

In various embodiments, the monitoring apparatus 100 may include the user interface 180 that has one or more buttons 185 for collecting one or more inputs from the person, the patient, a health care professional, or a caretaker. In various embodiments, the one or more buttons 185 are configured for activating or inactivating a function of the monitoring apparatus 100 or another system functionality, for communicating with a health care professional or caretaker, or for storing timestamps of events, such as, for example but not limited to identifying a bed time, a rise time, or a bed exit.

In various embodiments, the monitoring apparatus 100 may include one or more LED lights 190 to provide a status indicator of the apparatus, wherein the status indicator indicates a status of power, connectivity, configuration, or a status of measured data from a list of sleep quality, respiration signal quality, or successful data collection.

In radar-based devices, such as the monitoring apparatus 100, although potentially accurate for respiration monitoring, can be based on continuous wave doppler radar architectures. Continuous wave Doppler radar may not be able to distinguish between signals recorded at different distances to the device (for example, two persons in bed). Radar-based devices can also be based on pulsed radar. Time-gating (or range-gating) can be applied to pulsed radar to limit the detection range to the specific distance where the patient is expected to be. When applying time-gating in hardware, signals originating from distances outside of the detection range may be filtered out completely. The disadvantage of applying time-gating in hardware is that it may not be possible to monitor the patient when they reposition to a different distance to the device. This may happen in a home-scenario, as people move around furniture, and move around their house. In addition, when using continuous wave radar, it is not possible to simultaneously monitor a patient and their bed partner. Simultaneous two-person monitoring would allow for distinguishing between physiological signals originating from different people, with improved signal separation capabilities.

Optical monitoring systems require direct line of sight, light, and are often perceived as violating a patient's privacy. In addition, video data processing is computationally expensive. The present disclosure includes an apparatus for non-contact acquisition of human physiological data and environmental data, methods for on-device signal extraction, methods for transmission to remote storage and processing, methods for data analysis, and methods for long term monitoring of patients in a health care setting as well as notification and alert methods.

The present disclosure aids substantially in patient monitoring, by improving contactless access to multiple physiological and behavioral variables. Implemented on an apparatus in communication with a remote processor, the vital sign monitoring system disclosed herein provides practical touchless physiological and behavioral monitoring. This improved patient monitoring transforms a limited, uncomfortable, and uncertain monitoring process into one that happens seamlessly, without the normally routine need for the patient to play an active role in the monitoring. This unconventional approach improves the functioning of the clinical or home health care environment, by allowing local or remote health care providers ready access to physiological and behavioral variables.

The monitoring apparatus and system may be implemented as a series of monitored or computed variables, viewable on a display, and operated by a control process executing on a processor that accepts user inputs from a keyboard, mouse, or touchscreen interface, and that is in communication with one or more remote processors. In that regard, the control process performs certain specific operations in response to different inputs or selections made at different times.

These descriptions are provided for exemplary purposes and should not be considered to limit the scope of the vital sign monitoring system. Certain features may be added, removed, or modified without departing from the spirit of the claimed subject matter.

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

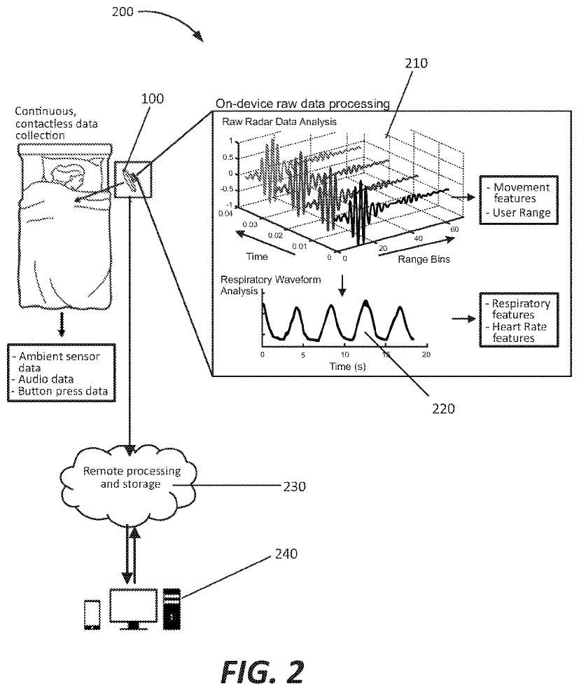

FIG. 2 is a schematic overview of a monitoring system 200, in accordance with various embodiments of the present disclosure. FIG. 2 shows a schematic of the entire monitoring system 200 which employs, for example, the monitoring apparatus 100 as described with respect to FIG. 1. The monitoring system 200 acquires raw data 210 (e.g., motion and proximity data) via the monitoring apparatus 100 when a person in a bed is being monitored by the monitoring system 200. The raw data is 210 is then analyzed or processed to obtain processed data 220, which is illustrated as a waveform data. The processing is done via a processor (e.g., the processor 120) of the monitoring apparatus 100 to determine one or more physiological and/or behavior features of the person being monitored. In some embodiments, the raw data can be transmitted to a remote process and storage 230 or a remote device 240 for further processing or analysis.

FIG. 3A is a perspective view of the monitoring apparatus 100 placed beside a bed, in a home setting, in accordance with various embodiments of the present disclosure. As illustrated in FIG. 3A, the monitoring apparatus 100 is intended for continuous, non-contact data collection of a person or patient in bed or in the vicinity of the monitoring apparatus 100. The monitoring apparatus 100 is typically placed beside the bed, ensuring that the person or the patient is within the apparatus' detection range, e.g., from 0.01 m to 30 m. The monitoring apparatus 100 is intended for health monitoring and may be used in a home setting (e.g., remote patient monitoring). For monitoring of a person in bed, the monitoring apparatus 100 may be placed on a nightstand. The monitoring apparatus 100 may also be attached to the bed, to the wall, to the ceiling, or underneath the bed. The monitoring apparatus 100 may also be integrated within the bed. The monitoring apparatus 100 may be used to monitor the patient throughout a room or accommodation and may thus be placed anywhere in a living or care facility. Multiple instances of the monitoring apparatus 100 may be used to monitor one or more persons or patients as they move around a living space or healthcare facility.

FIG. 3B is a perspective view of the monitoring apparatus 100 placed beside a bed, in a healthcare setting, in accordance with various embodiments of the present disclosure. The monitoring apparatus 100 is typically placed beside the bed, ensuring that the patient is in the apparatus' detection range. The apparatus may be used in a healthcare facility (e.g., hospital, skilled nursing facility, rehabilitation center, care home, etc.).

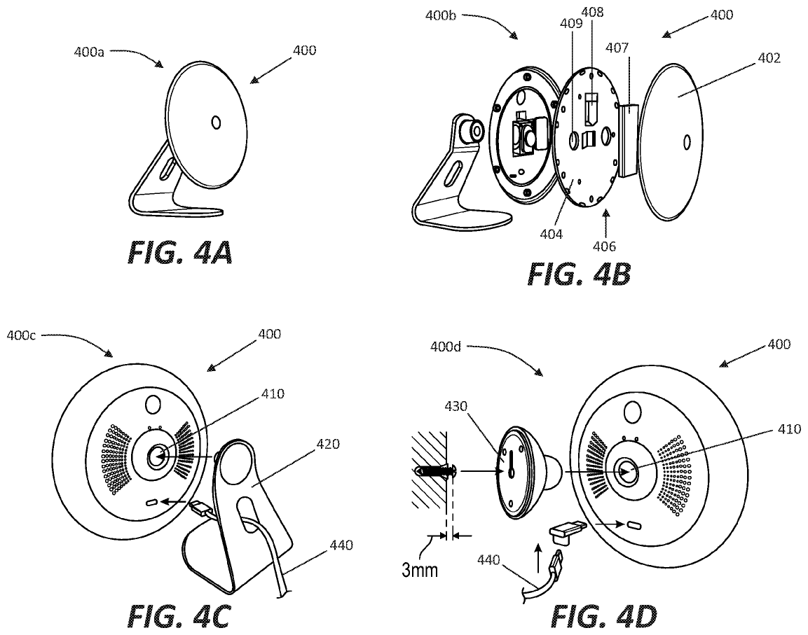

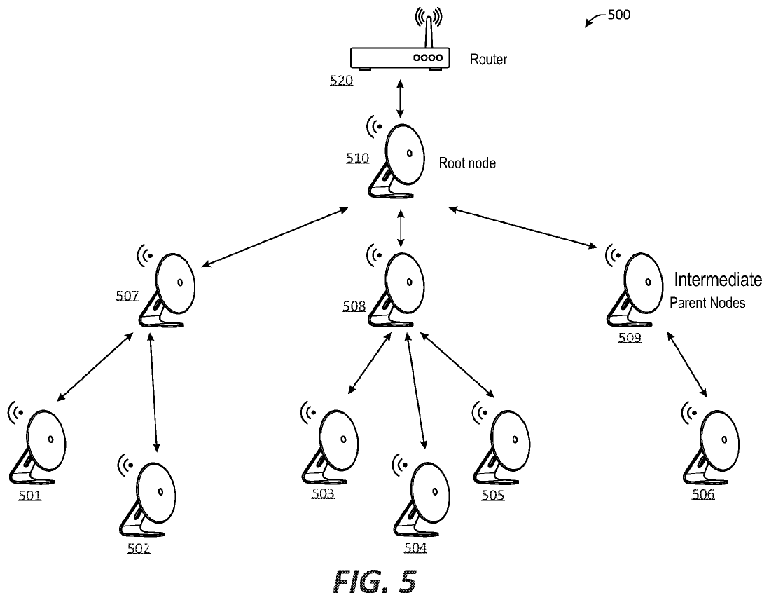

FIG. 4A is a perspective view 400a of a monitoring apparatus 400, in accordance with various embodiments of the present disclosure. FIG. 4B is an exploded view 400b of the monitoring apparatus 400, in accordance with various embodiments of the present disclosure. FIG. 4C is a perspective view 400c illustrating the monitoring apparatus 400 with a magnetic connector 410 to a stand 420, in accordance with various embodiments of the present disclosure. FIG. 4D is a perspective view 400d illustrating the magnetic connector 410 of the monitoring apparatus 400 to a wall mount 430, in accordance with various embodiments of the present disclosure. In various embodiments, the monitoring apparatus 400 is similar or the same as the monitoring apparatus 100 of FIG. 1.

As illustrated in views 400a and 400b, respectively of FIGS. 4A and 4B, the monitoring apparatus 400 includes a protective casing 402 containing a printed circuit board (PCB) 404. The PCB 404 may include a plurality of components 406, including but not limited to one or more sensor components, such as a radar system 407, one or more processing components, such as a processor 408, one or more storage components, one or more communication components, such as a transmitter 409, actuator components, and/or power supply components.

As illustrated in views 400c and 400d, respectively of FIGS. 4C and 4D, the magnetic connector 410 may be used to connect the main body of the monitoring apparatus 400 to the stand 420 or a mounting mechanism, such as the wall mount 430. A mounting mechanism may be connected to the wall, to the bed, to other healthcare equipment, or other furniture.

In various embodiments, the processor 408 is similar or the same as the processor 120 as described with respect to FIG. 1. The processor 408 may include any combination of general-purpose computing devices, reduced instruction set computing (RISC) devices, application-specific integrated circuits (ASICs), field programmable gate arrays (FPGAs), or other related logic devices, including mechanical and quantum computers. In some embodiments, the processor 408 includes a memory in which instructions or information are stored, and the processor 408 operates based on the instructions or information. The memory may be co-located on the same PCB 404 or another component, such as a chip with processing elements or else located external to a board or chip containing processing elements. The memory may comprise any combination of read-only memory (ROM), programmable read-only memory (PROM), electrically erasable read-only memory (EEPROM), magnetic or electronic random access memory (RAM), flash memory, disk or tape drive, or other related memory types.

In various embodiments, the communication (including but not limited to software updates, firmware updates, or readings from the device) to and from the monitoring apparatus 400 can be accomplished using any suitable wireless or wired communication technology, such as a cable interface such as a USB, micro USB, Lightning, or FireWire interface, Bluetooth, Wi-Fi, ZigBee, Li-Fi, or cellular data connections such as 2G/GSM, 3G/UMTS, 4G/LTE/WiMax, or 5G. For example, a Bluetooth Low Energy (BLE) radio can be used to establish connectivity with a cloud service, for transmission of data, and for receipt of software patches. In some embodiments, a controller may be configured to communicate with a remote server, or a local device such as a laptop, tablet, or handheld device, or may include a display capable of showing status variables and other information.

In various embodiments, the communication, if any, within or between the components of the monitoring apparatus 400 may be through numerous methods or protocols. Serial communication protocols may include but are not limited to SPI, I²C, RS-232, RS-485, CAN, Ethernet, ARINC 429, MODBUS, MIL-STD-1553, or any other suitable method or protocol. Parallel protocols including but not limited to ISA, ATA, SCSI, PCI, IEEE-488, IEEE-1284, and other suitable protocols. Where appropriate, serial and parallel communications may be bridged by a UART, USART, or another appropriate subsystem.

In some embodiments, instead of a radiofrequency based remote sensing modality, the monitoring apparatus 400 can be configured to detect motion and range monitoring functionality though alternative remote sensors. In some embodiments, an ultrasound-based sensor may be used, or an optical sensor (video, infrared, laser), or a capacitive sensor. Alternatively, a ‘semi-contact’ sensor such as an accelerometer or pressure sensor may be used when the apparatus is connected to the bed or mattress of the patient. In this case, presence, motion (and derived respiration and heart activity) can be obtained, but user distance to the apparatus cannot be determined when the user is out of bed.

In some embodiments, the monitoring apparatus 400 can be configured with light or ambient sensors, such as light sensor 170. A light sensor (e.g., a red-green-blue or RGB light sensor) may be used to measure light levels in the room. A microphone, such as the microphone 150, may be used to measure ambient noise levels in the room. Additional ambient sensors may include a temperature sensor, humidity sensor, or air quality sensor. Ambient sensor data may be used to analyze user behavior, estimate sleep behavior, and analyze bedroom quality. An apparatus microphone may be used to record audio data, which may be further processed for respiratory analysis, in conjunction with remote sensor (radar) respiration data. A thermographic camera may be employed by the apparatus to collect nocturnal video data of a sleeping patient, or to determine body temperature of the patient.

In some embodiments, the monitoring apparatus 400 may include buttons, such as buttons 185 of the user interface 180, to register an input from a user, a person, a patient, a healthcare professional or a caretaker. Alternatively, other sensors may be used, such as a capacitive touch sensor. In some embodiments, the monitoring apparatus 400 may also include a speaker, such as the speaker 160, to provide user feedback, through sounds and/or spoken word. The combination of speaker and microphone may be used in combination with voice assistant technology. The voice assistant in this case may be used specifically for telemedicine purposes, such as performing a symptom check, or for reminding a patient of their prescribed therapy or intervention. The speaker and microphone may also be used for direct communication with healthcare professionals or caregivers.

In some embodiments, the monitoring apparatus 400 may include indicator lights (e.g., RGB LED indicator lights or one or more LED lights 190), that may, for example, be organized in a circular arrangement on the front of the device. Other arrangements or locations may be used instead or in addition. Indicator lights may for example inform the user of connectivity status, power status, mode (configuration or monitoring), etc. Indicator lights may also be used to provide feedback to users on specific functions of the overall system. For example, when the person or patient triggers a spot measurement of respiratory rate, indicator lights may indicate once a spot measurement has been completed. For sleep monitoring functionality, indicator lights may indicate the start and end of a sleep session, as well as provide feedback on the sleep quality after a sleep session has been analyzed. The intensity or brightness of the indicator lights may be adaptive to the ambient light levels, such that LEDs on the monitoring apparatus 400 do not disturb the person or the patient in low light conditions (during sleep), although they may be visible during the day. In a non-limiting example, indicator lights on the apparatus may be disabled by the user by a press of the button on the monitoring apparatus 400.

Also illustrated in FIG. 4C, the monitoring apparatus 400 is powered by a power supply cable 440, connected to a power source (not shown). The monitoring apparatus 400 may also have a built-in battery (not shown), to facilitate device functioning for limited duration without the need for power from the power source. The monitoring apparatus 400 may also have internal memory for limited data storage, in case an interruption of data transmission occurs. The monitoring apparatus 400 may also have an internal clock with accompanying battery to be time-aware during absence of internet connectivity.

In accordance with various embodiments, the monitoring apparatus 400 is configured to collect multimodal sensor data continuously. Such data may be stored locally on the monitoring apparatus 400, for monitoring scenarios when communication with a remote server is not possible.

FIG. 5 is a schematic view of a plurality of monitoring apparatuses 501-510 in a mesh network 500, in accordance with various embodiments of the present. As illustrated in FIG. 5, the plurality of monitoring apparatuses 501-510 are formed in the mesh network 500 and connected to a router 520. As healthcare professionals increasingly rely on continuous patient monitoring, it becomes problematic that many healthcare institutions (e.g., skilled nursing facilities), do not have a connectivity infrastructure such as facility-wide Wi-Fi coverage. This may complicate deployment of medical monitoring technologies. Additionally, for remote patient monitoring, installation of devices is complicated by the fact that not all potential users (often elderly patients) have Wi-Fi or a smartphone, and often are not skilled to configure a device to a local network.

In accordance with various embodiments, continuous monitoring technologies and alerting systems may rely on continuous data transmission. Connectivity of medical devices may be achieved using Wi-Fi, or direct connectivity to a ‘hub’ device. Consumer devices as well as medical devices intended for the home environment often rely on Wi-Fi, or connect to the user mobile phone, e.g. via Bluetooth. In accordance with various embodiments, monitoring solutions assume wide and reliable Wi-Fi network coverage, and a level of technological know-how of the user. This makes current solutions unsuitable for deployment in many homes or healthcare facilities.

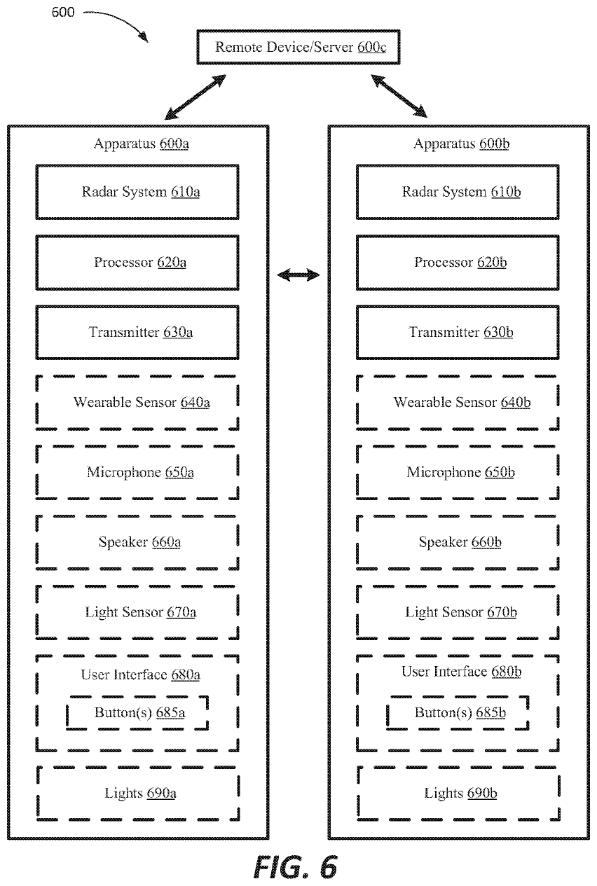

In an example, data obtained or generated by the vital sign monitoring system may be transmitted from the apparatus to a remote server for data processing and/or storage. Raw sensor data, as well as data processed on the apparatus by embedded algorithms may be transmitted. Data may be transmitted by connection to a local Wi-Fi network. Each individual apparatus may be connected to a router with active internet connection through Wi-Fi directly. Alternatively, when multiple instances of the apparatus are installed in the same facility, and Wi-Fi coverage is limited, a mesh network may be created. A schematic illustration of such a mesh network is shown in FIG. 6.

In an example, each device can connect to a Wi-Fi access point directly. If such a connection is not possible or not successful, two or more monitoring apparatuses from the plurality of monitoring apparatuses 501-510 may form the mesh network 500 allowing peer to peer communication. In this configuration, a single piece of apparatus must function as the root node and be connected to a Wi-Fi access point, such as the router 520. All other monitoring apparatuses in the mesh network 500 may act as intermediate parent nodes and may for example connect to up to 10 other monitoring apparatuses. The mesh network 500 of apparatus connectivity allows monitoring of patients outside of a Wi-Fi access point coverage. In addition, this newly created network can be used as an interface for other medical monitoring instruments that wouldn't otherwise be deployable due to a lack of infrastructure.

In various embodiments, data may also be transmitted to a remote server, such as the remote device 105 via one or more cellular networks. This solution is particularly suitable for deployment at a person or patient's home and does not require any device configuration by the person or patient. Data may also be transmitted directly to a local device such as computer, tablet, or mobile phone, using either cable or wireless connectivity. When the vital sign monitoring system is transmitting data to a local device, data storage and processing may be performed on the local device, or raw data may be transmitted further to a remote server. In various embodiments, data also be transmitted by all previously mentioned means to a local ‘hub’, collecting data of multiple monitoring apparatuses simultaneously, after which data can be transmitted to a remote server or other digital environment. In some embodiments, one or more of the monitoring apparatuses 501-510 may contain internal memory to temporarily store data on the apparatus, in case of a temporary loss of data transmission.

In various embodiments, one of the monitoring apparatuses 501-510 may communicate with other monitoring apparatuses in its vicinity, and act as a data collection and transmission hub for various external devices. Examples of external devices that may be paired with the apparatus are wearable or nearable sensors and monitors, such as pulse oximeters, heart rate monitors, thermometers, pressure sensors, optical sensors, capacitive sensors, or environmental sensors. External devices may communicate with the apparatus via wireless communication, such as Bluetooth or Wi-Fi. The apparatus may send data to external devices or trigger a measurement. The apparatus may receive measurement or status data from external devices. The apparatus may process received data, store received data, and/or transmit received data to a remote server or a local device such as a laptop, tablet, or handheld device. The apparatus may include a display capable of showing received and/or processed data.

FIG. 6 is a schematic illustration of a monitoring system 600 showing interconnectivity between two monitoring apparatuses 600a and 600b with a remote device/server 600c, in accordance with various embodiments of the present disclosure. The monitoring apparatuses 600a and 600b are similar to one or more of the monitoring apparatuses 501-510 or the monitoring apparatus 100. As shown in FIG. 6, the monitoring apparatus 600a includes a radar system 610a, a processor 620a, and a transmitter 630a. In accordance with various embodiments, the monitoring apparatus 600a can include one or more of a wearable sensor 640a, a microphone 650a, a speaker 660a, a light or ambient sensor 670a, a user interface 680a having one or more buttons 685a, or one or more LED lights 690a. In various embodiments, the monitoring apparatus 600a is configured to send data/information to the remote device/server 600c (or a storage unit). Similarly, the monitoring apparatus 600b includes a radar system 610b, a processor 620b, and a transmitter 630b. In accordance with various embodiments, the monitoring apparatus 600b can include one or more of a wearable sensor 640b, a microphone 650b, a speaker 660b, a light or ambient sensor 670b, a user interface 680b having one or more buttons 685b, or one or more LED lights 690b. In various embodiments, the monitoring apparatus 600b is configured to send data/information to the remote device/server 600c (or a storage unit).

As illustrated in FIG. 6, the monitoring system 600 is configured for monitoring a plurality of patients (or persons). The monitoring system 600 includes a plurality of apparatuses, such as the monitoring apparatuses 600a and 600b or the plurality of monitoring apparatuses 501-510, in a mesh network, such as the mesh network 500. In accordance with various embodiments, the monitoring apparatuses 600a and 600b (in the mesh network) are configured for sharing data within the mesh network and/or with the remote device/server 600c. The monitoring system 600 includes at least the monitoring apparatus 600a and the monitoring apparatus 600b). In various embodiments, the monitoring apparatus 600a is positioned at a first location within a first local area and the monitoring apparatus 600b is positioned at a second location within a second local area.

In various embodiments, the monitoring apparatus 600a includes the radar system 610a configured for acquiring motion and proximity data of at least one patient from the plurality of patients. This acquisition of motion and proximity data of the patient can be performed when monitoring the patient at a plurality of distances from the monitoring apparatus 600a. The radar system 610a is similar to or identical to the radar system 110 and therefore, the radar system 610a will not be described in further detail. In various embodiments, the monitoring apparatus 600a includes the processor 620a configured for processing the acquired motion and proximity data to identify one or more physiological and/or behavioral features of at least one patient from the plurality of patients. The processor 620a is similar to or identical to the processor 120 and therefore, the processor 620a will not be described in further detail. In various embodiments, the monitoring apparatus 600a includes the transmitter 630a configured for transmitting the one or more physiological and/or behavioral features of at least one patient from the plurality of patients to the monitoring apparatus 600b in the mesh network 600 or the remote device/server 600c. The transmitter 630a is similar to or identical to the transmitter 130 and therefore, the transmitter 630a will not be described in further detail.

In various embodiments, the transmitter 630a is configured for transmitting the acquired motion and proximity data (raw acquired data) of at least one patient from the plurality of patients to monitoring apparatus 600b in the mesh network 600 or the remote device/server 600c.

In various embodiments, the radar system 610a includes a coherent pulsed ultra-wide band radar. In various embodiments, the radar system 610a is a multistatic radar system configured for acquiring motion and proximity data of at least the first patient from the plurality of patients. In various embodiments, the multistatic radar system is configured for beamforming to direct a radar beam to one or more specific anatomical portions of at least one patient from the plurality of patients. In various embodiments, the multistatic radar system is configured for capturing a position of at least one patient from the plurality of patients at the plurality of distances.

In various embodiments, the radar system 610a is configured for acquiring the motion and proximity data of at least one patient from the plurality of patients at the plurality of distances between 0.01 m and 30 m from the monitoring apparatus 600a. In various embodiments, the radar system 610a is configured for acquiring the motion and proximity data of at least one patient from the plurality of patients at the plurality of distances between 0.3 m and 3.2 m from the monitoring apparatus 600a.

In various embodiments, the acquired motion and proximity data of at least one patient from the plurality of patients are processed by the processor 620a for identifying at least one patient from other patients present between 0.01 m and 30 m from the monitoring apparatus 600a. In various embodiments, the acquired motion and proximity data of at least one patient from the plurality of patients are processed by the processor 620a for identifying at least one patient from other patients present between 0.3 m and 3.2 m from the monitoring apparatus 600a.

In various embodiments, the acquired motion and proximity data includes respiratory-induced body movements from thoracic and abdominal areas of at least one patient from the plurality of patients. In various embodiments, the acquired motion and proximity data includes vital signs of at least one patient from the plurality of patients. In various embodiments, the acquired motion and proximity data is used to determine a breathing pattern or monitor a heart activity of at least the first patient from the plurality of patients.

In various embodiments, the acquired motion and proximity data is used to monitor behavior of at least one patient from the plurality of patients, and wherein the behavior includes one of bed occupancy, activity, or sleep behavior. In various embodiments, the one or more behavioral features include sleep behavior of at least one patient from the plurality of patients. In various embodiments, the sleep behavior includes a pattern of sleep stages that at least one patient from the plurality of patients goes through during sleep. In various embodiments, the acquired motion and proximity data includes data captured while at least one patient from the plurality of patients is asleep, awake in bed, moves out of a bed, moves into a bed, falls down, or moves around in a vicinity of the monitoring apparatus 600a.

As illustrated in FIG. 6, the monitoring apparatus 600b includes the radar system 610b and is positioned at a second location within a second local area. The radar system 610b is configured for acquiring motion and proximity data of a second patient from the plurality of patients. In various embodiments, the monitoring apparatus 600b further includes the processor 620b configured for processing the acquired motion and proximity data of the second patient to identify one or more physiological and/or behavioral features of the second patient. In various embodiments, the transmitter 630b is configured for transmitting the one or more physiological and/or behavioral features of the second patient to the monitoring apparatus 600a or the remote device/server 600c.

As illustrated in FIG. 6, the wearable sensor 640a is one from a list of a pulse-oximeter, a heart rate monitor, an ultrasound sensor, or a thermometer, or a nearable sensor from a list of an accelerometer, a pressure sensor, optical sensor, including a video-, infrared- or laser-based sensor, a capacitive or touch sensor, or an environmental sensor.

In various embodiments, the monitoring apparatus 600a is configured as a hub for collecting sensor data from one or more of the wearable sensor or nearable sensor and transmitting the collected sensor data to the monitoring apparatus 600b or the remote device/server 600c.

In various embodiments, the microphone 650a and the speaker 660a are configured for communicating with a health care professional or caretaker. In various embodiments, the microphone 650a is used for monitoring a physiological function of at least one patient from the plurality of patients and the physiological function includes one of respiration, coughing, or snoring. In various embodiments, the microphone 650a is used for monitoring noise in an environment of at least one patient from the plurality of patients. In various embodiments, the microphone 650a is used for monitoring a behavior of at least one patient from the plurality of patients and the behavior includes one of TV watching, going to bed, or falling down. In various embodiments, the light sensor 670a is configured for monitoring a light level of an environment of at least one patient from the plurality of patients. In various embodiments, the light sensor 670a is used for monitoring a bed time of at least the first patient from the plurality of patients. In various embodiments, the bed time is determined when the light sensor 670a detects that a light in the environment of at least one patient from the plurality of patients is turned off.

In various embodiments, the transmitter 630a is a wired communication component configured to work over Ethernet or USB protocol. In various embodiments, the transmitter 630a is a wireless communication component configured to work over Bluetooth, Wi-Fi, a local area network, a wide area network, or a cellular network.

In various embodiments, the user interface 680a includes one or more buttons 685a for collecting one or more inputs from at least one patient or a healthcare professional or a caretaker. In various embodiments, one or more buttons are 685a are configured for activating or inactivating a functionality of the monitoring apparatus 600a, for communicating with a health care professional or caretaker, or for storing timestamps of events identifying a bed time, a rise time, or a bed exit.

In various embodiments, the one or more LED lights 690a is configured to provide a status indicator of the first apparatus, wherein the status indicator indicates a status of power, connectivity, configuration, or a status of measured data from a list of sleep quality, respiration signal quality, or successful data collection.

In various embodiments, the various apparatuses, systems, and methods for described herein, can be implemented via computer software or hardware and various components can be connected via a direct connection or through an internet connection.

It should be appreciated that the various engines and features depicted in various system and method embodiments herein can be combined or collapsed into a single engine, component or module, depending on the requirements of the particular application or system architecture. Moreover, in various embodiments, the systems can comprise additional engines or components as needed by the particular application or system architecture.

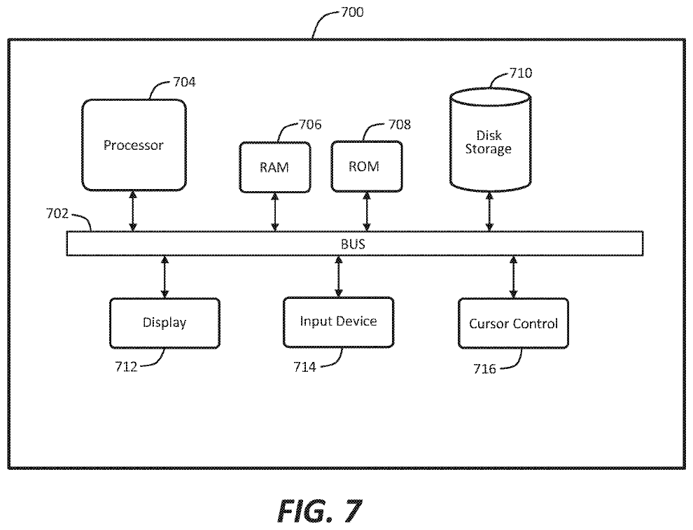

FIG. 7 is a block diagram illustrating a computer system 700 upon which embodiments of the present teachings may be implemented. In various embodiments of the present teachings, computer system 700 can include a bus 702 or other communication mechanism for communicating information and a processor 704 coupled with bus 702 for processing information. In various embodiments, computer system 700 can also include a memory, which can be a random-access memory (RAM) 706 or other dynamic storage device, coupled to bus 702 for determining instructions to be executed by processor 704. Memory can also be used for storing temporary variables or other intermediate information during execution of instructions to be executed by processor 704. In various embodiments, computer system 700 can further include a read only memory (ROM) 708 or other static storage device coupled to bus 702 for storing static information and instructions for processor 704. A storage device 710, such as a magnetic disk or optical disk, can be provided and coupled to bus 702 for storing information and instructions.

In various embodiments, computer system 700 can be coupled via bus 702 to a display 712, such as a cathode ray tube (CRT) or liquid crystal display (LCD), for displaying information to a computer user. An input device 714, including alphanumeric and other keys, can be coupled to bus 702 for communication of information and command selections to processor 704. Another type of user input device is a cursor control 716, such as a mouse, a trackball or cursor direction keys for communicating direction information and command selections to processor 704 and for controlling cursor movement on display 712. This input device 714 typically has two degrees of freedom in two axes, a first axis (i.e., x) and a second axis (i.e., y), that allows the device to specify positions in a plane. However, it should be understood that input devices 714 allowing for 3-dimensional (x, y and z) cursor movement are also contemplated herein.

Consistent with certain implementations of the present teachings, results can be provided by computer system 700 in response to processor 704 executing one or more sequences of one or more instructions contained in memory 706. Such instructions can be read into memory 706 from another computer-readable medium or computer-readable storage medium, such as storage device 710. Execution of the sequences of instructions contained in memory 706 can cause processor 704 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 704 for execution. Such a medium can take many forms, including but not limited to, non-volatile media, volatile media, and transmission media. Examples of non-volatile media can include, but are not limited to, dynamic memory, such as memory 706. Examples of transmission media can include, but are not limited to, coaxial cables, copper wire, and fiber optics, including the wires that comprise bus 702.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 704 of computer system 700 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 700 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 700, whereby processor 704 would execute the analyses and determinations provided by these engines, subject to instructions provided by any one of, or a combination of, memory components 706/708/710 and user input provided via input device 714.

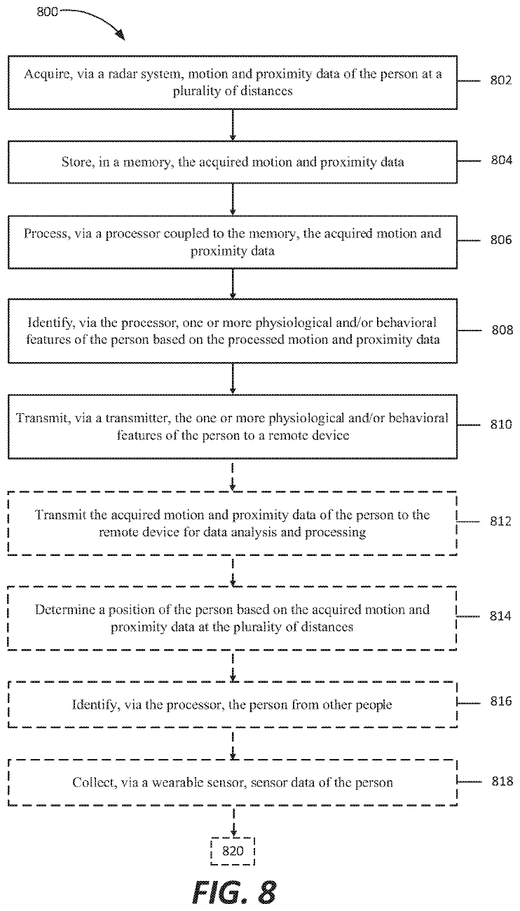

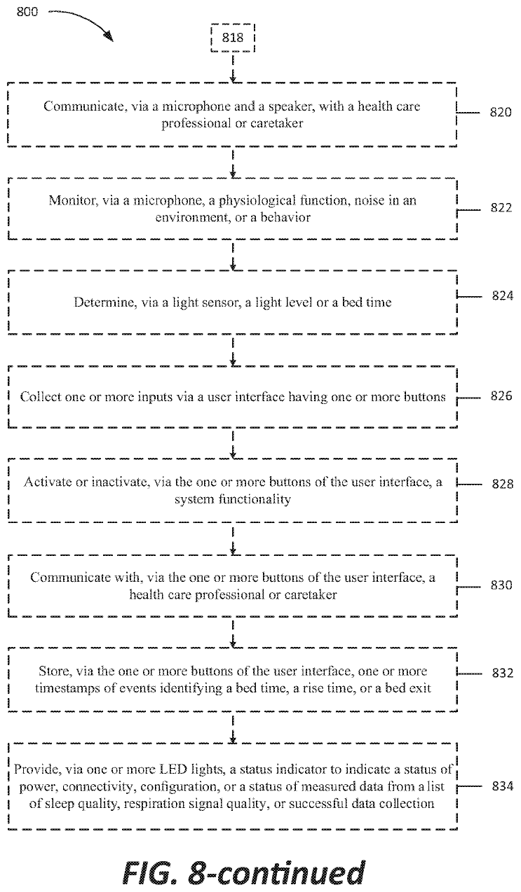

Referring now to FIG. 8, a process flow for a method 800 of non-contact monitoring a person is described, in accordance with various embodiments of the present disclosure. The method 800 includes at step 802, acquiring, via a radar system, motion and proximity data of the person at a plurality of distances. The radar system is similar or identical to the radar system 110 and therefore will not be described in further detail. In various embodiments, the radar system is a coherent pulsed ultra-wide band radar. In various embodiments, the radar system is a multistatic radar system configured for acquiring motion and proximity data of a plurality of persons. In various embodiments, the multistatic radar system is configured for beamforming to direct a radar beam to one or more specific anatomical portions of at least one person of the plurality of persons.

At step 804, the method 800 includes storing, in a memory, the acquired motion and proximity data. The method 800 includes, at step 806, processing, via a processor coupled to the memory, the acquired motion and proximity data. The processor is similar or identical to the processor 120 and therefore will not be described in further detail.

The method 800 includes, at step 808, identifying, via the processor, one or more physiological and/or behavioral features of the person based on the processed motion and proximity data, and at step 810, transmitting, via a transmitter, the one or more physiological and/or behavioral features of the person to a remote device. The transmitter is similar or identical to the transmitter 130 and therefore will not be described in further detail. In various embodiments, the transmitter is a wired communication component configured to work over Ethernet or USB protocol. In various embodiments, the transmitter is a wireless communication component configured to work over Bluetooth, Wi-Fi, a local area network, a wide area network, or a cellular network.

In various embodiments, the method 800 further includes transmitting the acquired motion and proximity data of the person to the remote device for data analysis and processing at step 812. In various embodiments, the method 800 includes, at step 814, determining a position of the person based on the acquired motion and proximity data at the plurality of distances. In various embodiments, the radar system is configured for acquiring the motion and proximity data of the person at the plurality of distances between 0.01 m and 30 m from the radar system. In various embodiments, the radar system is configured for acquiring the motion and proximity data of the person at the plurality of distances between 0.3 m and 3.2 m from the radar system.

In various embodiments, the method 800 further includes, at step 816, identifying, via the processor, the person from other people present between 0.01 m and 30 m or between 0.3 m and 3.2 m from the radar system. In various embodiments, the acquired motion and proximity data includes respiratory-induced body movements from thoracic and abdominal areas of the person. In various embodiments, the acquired motion and proximity data includes vital signs of the person. In various embodiments, the acquired motion and proximity data is used to determine a breathing pattern or to monitor a heart activity of the person. In various embodiments, the acquired motion and proximity data is used to monitor behavior of the person, and wherein the behavior comprises one of bed occupancy, activity, or sleep behavior.

In various embodiments, the one or more behavioral features of the person includes sleep behavior of the person. In various embodiments, the sleep behavior of the person includes a pattern of sleep stages that the person goes through during sleep. In various embodiments, the acquired motion and proximity data includes data captured while the person is asleep, awake in bed, moves around in a vicinity of the radar system, moves out of a bed, moves into a bed, or falls down. In various embodiments, the method 800 includes transmitting, via the transmitter, the one or more physiological and/or behavioral features of the person to one or more apparatuses in a mesh network. In various embodiments, the method 800 includes sharing data with the one or more apparatuses in the mesh network.

In various embodiments, the method 800 includes, at step 818, collecting, via a wearable sensor, sensor data of the person, wherein the wearable sensor is one from a list of a pulse-oximeter, a heart rate monitor, an ultrasound sensor, or a thermometer, or from a nearable sensor from a list of an accelerometer, a pressure sensor, optical sensor, including a video-, infrared- or laser-based sensor, a capacitive or touch sensor, or an environmental sensor. In various embodiments, the method 800 includes transmitting the collected sensor data to the remote device.

In various embodiments, the method 800 includes, at step 820, communicating, via a microphone and a speaker, with a health care professional or caretaker. In various embodiments, the method 800 includes, at step 822, monitoring, via a microphone, a physiological function, noise in an environment, or a behavior of the person. In various embodiments, the physiological function includes one of respiration, coughing, or snoring. In various embodiments, the behavior includes one of TV watching, going to bed, or falling down.

In various embodiments, the method 800 includes, at step 824, monitoring, via a light sensor, a light level of an environment of the person or a bed time of the person. In various embodiments, the bed time is determined when the light sensor detects that a light in an environment of the person is turned off.

In various embodiments, the method 800 includes, at step 826, collecting one or more inputs via a user interface having one or more buttons. In various embodiments, the method 800 includes, at step 828, activating or inactivating, via the one or more buttons of the user interface, a system functionality. In various embodiments, the method 800 includes, at step 830, communicating with, via the one or more buttons of the user interface, a health care professional or caretaker. In various embodiments, the method 800 includes, at step 832, storing, via the one or more buttons of the user interface, one or more timestamps of events identifying a bed time, a rise time, or a bed exit.

In various embodiments, the method 800 includes, at step 834, providing, via one or more LED lights, a status indicator to indicate a status of power, connectivity, configuration, or a status of measured data from a list of sleep quality, respiration signal quality, or successful data collection.

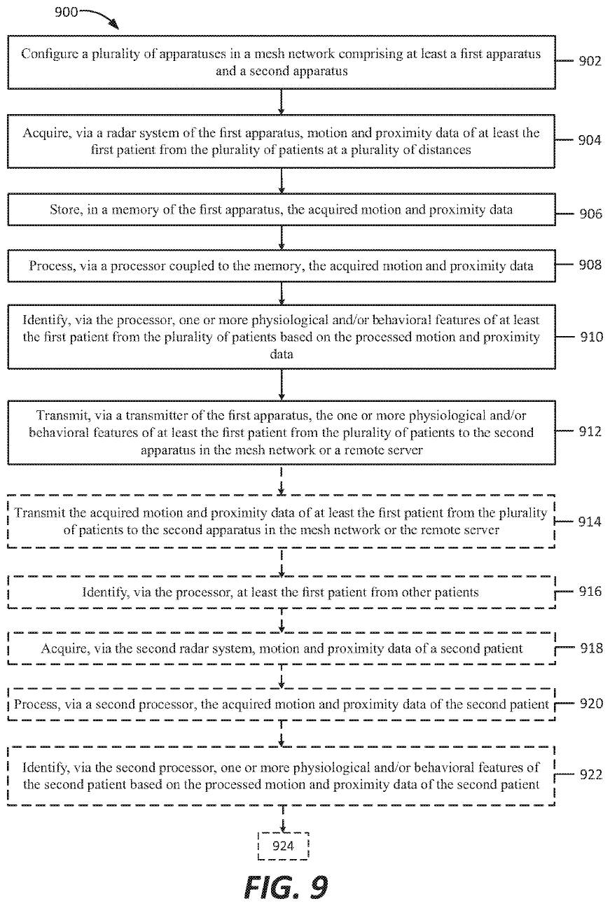

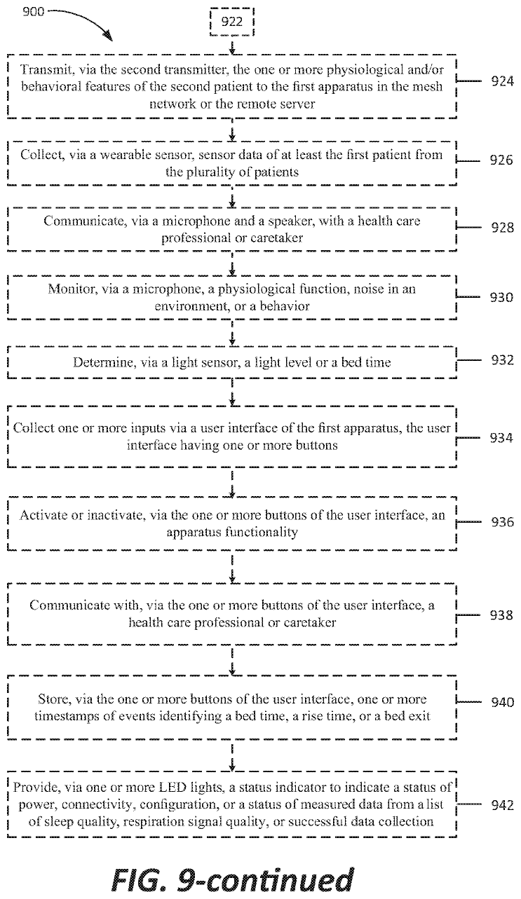

FIG. 9 is a process flow for a method 900 of monitoring a plurality of patients, in accordance with various embodiments of the present disclosure. The method 900 includes at step 902, configuring a plurality of apparatuses (e.g., monitoring apparatuses 100, 600a or 600b) in a mesh network comprising at least a first apparatus (e.g., monitoring apparatus 600a) and a second apparatus (e.g., monitoring apparatus 600b). In various embodiments, the plurality of apparatuses in the mesh network are configured for sharing data within the mesh network and/or with a remote server. In various embodiments, the first apparatus is positioned at a first location within a first local area, wherein at least a first patient from the plurality of patients is present at the first location and being monitored by the first apparatus.

As illustrated in FIG. 9, the method 900 includes, at step 904, acquiring, via a radar system of the first apparatus, motion and proximity data of at least the first patient from the plurality of patients at a plurality of distances. The radar system is similar or identical to the radar system 110 and therefore will not be described in further detail. In various embodiments, the radar system is a coherent pulsed ultra-wide band radar. In various embodiments, the radar system is a multistatic radar system configured for acquiring motion and proximity data of at least the first patient from the plurality of patients. In various embodiments, the multistatic radar system is configured for beamforming to direct a radar beam to one or more specific anatomical portions of at least the first patient from the plurality of patients.

At step 906, the method 900 includes storing, in a memory of the first apparatus, the acquired motion and proximity data. The method 900 includes, at step 908, processing, via a processor coupled to the memory, the acquired motion and proximity data. The processor is similar or identical to the processor 120 and therefore will not be described in further detail.

The method 900 includes, at step 910, identifying, via the processor, one or more physiological and/or behavioral features of at least the first patient from the plurality of patients based on the processed motion and proximity data, and at step 912, transmitting, via a transmitter of the first apparatus, the one or more physiological and/or behavioral features of at least the first patient from the plurality of patients to the second apparatus in the mesh network or a remote server. The transmitter is similar or identical to the transmitter 130 and therefore will not be described in further detail. In various embodiments, the transmitter is a wired communication component configured to work over Ethernet or USB protocol. In various embodiments, the transmitter is a wireless communication component configured to work over Bluetooth, Wi-Fi, a local area network, a wide area network, or a cellular network. In various embodiments, the method 900 further includes, at step 914, transmitting the acquired motion and proximity data of at least the first patient from the plurality of patients to the second apparatus in the mesh network or the remote server.

In various embodiments, the radar system is configured for acquiring the motion and proximity data of at least the first patient from the plurality of patients at the plurality of distances between 0.01 m and 30 m from the first apparatus. In various embodiments, the radar system is configured for acquiring the motion and proximity data of at least the first patient from the plurality of patients at the plurality of distances between 0.3 m and 3.2 m from the first apparatus.

In various embodiments, the method 900 further includes, at step 916, identifying, via the processor, at least the first patient from other patients present between 0.01 m and 30 m or between 0.3 m and 3.2 m from the first apparatus.

In various embodiments, the acquired motion and proximity data includes respiratory-induced body movements from thoracic and abdominal areas of at least the first patient from the plurality of patients. In various embodiments, the acquired motion and proximity data comprises vital signs of at least the first patient from the plurality of patients. In various embodiments, the acquired motion and proximity data is used to determine a breathing pattern or to monitor a heart activity of at least the first patient from the plurality of patients.

In various embodiments, the acquired motion and proximity data is used to monitor behavior of at least the first patient from the plurality of patients, and wherein the behavior comprises one of bed occupancy, activity, or sleep behavior. In various embodiments, the one or more behavioral features of at least the first patient from the plurality of patients includes sleep behavior of at least the first patient from the plurality of patients. In various embodiments, the sleep behavior comprises a pattern of sleep stages that at least the first patient from the plurality of patients goes through during sleep. In various embodiments, the acquired motion and proximity data includes data captured while at least the first patient from the plurality of patients is asleep, awake in bed, moves around in a vicinity of the first apparatus, moves out of a bed, moves into a bed, or falls down.

In various embodiments, the second apparatus includes a second radar system and is positioned at a second location within a second local area, and the method 800 further includes, at step 918, acquiring, via the second radar system, motion and proximity data of a second patient. In various embodiments, the second apparatus further includes a second processor and a second transmitter, the method 800 further includes, at step 920, processing, via the second processor, the acquired motion and proximity data of the second patient, at step 922, identifying, via the second processor, one or more physiological and/or behavioral features of the second patient based on the processed motion and proximity data of the second patient, and at step 924, transmitting, via the second transmitter, the one or more physiological and/or behavioral features of the second patient to the first apparatus in the mesh network or the remote server.

In various embodiments, the method 900 further includes, at step 926, collecting, via a wearable sensor, sensor data of at least the first patient from the plurality of patients, wherein the wearable sensor is one from a list of a pulse-oximeter, a heart rate monitor, an ultrasound sensor, or a thermometer, or from a nearable sensor from a list of an accelerometer, a pressure sensor, optical sensor, including a video-, infrared- or laser-based sensor, a capacitive or touch sensor, or an environmental sensor. In various embodiments, the method 900 further includes transmitting the collected sensor data to the second apparatus or the remote server.

In various embodiments, the method 900 further includes, at step 928, communicating, via a microphone and a speaker, with a health care professional or caretaker. In various embodiments, the method 900 further includes, at step 930, monitoring, via a microphone, a physiological function, noise in an environment, or a behavior of at least the first patient from the plurality of patients. In various embodiments, the physiological function includes one of respiration, coughing, or snoring. In various embodiments, the behavior includes one of TV watching, going to bed, or falling down.

In various embodiments, the method 900 further includes, at step 932, monitoring, via a light sensor, a light level of an environment of at least the first patient from the plurality of patients. In various embodiments, the method 900 further includes monitoring, via a light sensor, a bed time of at least the first patient from the plurality of patients. In various embodiments, the bed time is determined when the light sensor detects that a light in an environment of at least the first patient from the plurality of patients is turned off.

In various embodiments, the method 900 further includes, at step 934, collecting one or more inputs via a user interface of the first apparatus, the user interface having one or more buttons. In various embodiments, the method 900 further includes, at step 936, activating or inactivating, via the one or more buttons of the user interface, an apparatus functionality. In various embodiments, the method 900 further includes, at step 938, communicating with, via the one or more buttons of the user interface, a health care professional or caretaker. In various embodiments, the method 900 further includes, at step 940, storing, via the one or more buttons of the user interface, one or more timestamps of events identifying a bed time, a rise time, or a bed exit.

In various embodiments, the method 900 further includes, at step 942, providing, via one or more LED lights of the first apparatus, a status indicator of the first apparatus to indicate a status of power, connectivity, configuration, or a status of measured data from a list of sleep quality, respiration signal quality, or successful data collection.

### RECITATION OF EMBODIMENTS

EMBODIMENT 1. An apparatus for non-contact monitoring of a person, comprising: a radar system configured for acquiring motion and proximity data of a person at a plurality of distances; a processor configured for processing the acquired motion and proximity data to identify one or more physiological and/or behavioral features of the person; and a transmitter configured for transmitting the one or more physiological and/or behavioral features of the person to a remote device.

EMBODIMENT 2. The apparatus of embodiment 1, wherein the transmitter is configured for transmitting the acquired motion and proximity data of the person to the remote device.

EMBODIMENT 3. The apparatus of any preceding embodiment, wherein the radar system comprises a coherent pulsed ultra-wide band radar.

EMBODIMENT 4. The apparatus of any preceding embodiment, wherein the radar system is a multistatic radar system configured for acquiring motion and proximity data of a plurality of persons.

EMBODIMENT 5. The apparatus of embodiment 4, wherein the multistatic radar system is configured for beamforming to direct a radar beam to one or more specific anatomical portions of at least one person of the plurality of persons.

EMBODIMENT 6. The apparatus of any preceding embodiment, wherein the radar system is configured for determining a position of the person based on the acquired motion and proximity data at the plurality of distances.

EMBODIMENT 7. The apparatus of any preceding embodiment, wherein the radar system is configured for acquiring the motion and proximity data of the person at the plurality of distances between 0.01 m and 30 m from the apparatus.

EMBODIMENT 8. The apparatus of any preceding embodiment, wherein the radar system is configured for acquiring the motion and proximity data of the person at the plurality of distances between 0.3 m and 3.2 m from the apparatus.

EMBODIMENT 9. The apparatus of embodiment 7, wherein the acquired motion and proximity data of the person are processed by the processor for identifying the person from other people present between 0.01 m and 30 m from the apparatus.

EMBODIMENT 10. The apparatus of embodiment 8, wherein the acquired motion and proximity data of the person are processed by the processor for identifying the person from other people present within the distance between 0.3 m and 3.2 m from the apparatus.

EMBODIMENT 11. The apparatus of any preceding embodiment, wherein the acquired motion and proximity data comprises respiratory-induced body movements from thoracic and abdominal areas of the person.

EMBODIMENT 12. The apparatus of any preceding embodiment, wherein the acquired motion and proximity data comprises vital signs of the person.

EMBODIMENT 13. The apparatus of any preceding embodiment, wherein the acquired motion and proximity data is used to determine a breathing pattern of the person.

EMBODIMENT 14. The apparatus of any preceding embodiment, wherein the acquired motion and proximity data is used to monitor a heart activity of the person.

EMBODIMENT 15. The apparatus of any preceding embodiment, wherein the acquired motion and proximity data is used to monitor behavior of the person, and wherein the behavior comprises one of bed occupancy, activity, or sleep behavior.

EMBODIMENT 16. The apparatus of any preceding embodiment, wherein the one or more behavioral features of the person comprises sleep behavior of the person.

EMBODIMENT 17. The apparatus of embodiment 16, wherein the sleep behavior comprises a pattern of sleep stages that the person goes through during sleep.

EMBODIMENT 18. The apparatus of any preceding embodiment, wherein the acquired motion and proximity data comprises data captured while the person is asleep.

EMBODIMENT 19. The apparatus of any preceding embodiment, wherein the acquired motion and proximity data comprises data captured while the person is awake in bed.

EMBODIMENT 20. The apparatus of any preceding embodiment, wherein the acquired motion and proximity data comprises data captured while the person moves around in a vicinity of the apparatus.

EMBODIMENT 21. The apparatus of any preceding embodiment, wherein the acquired motion and proximity data comprises data captured when the person moves out of a bed.

EMBODIMENT 22. The apparatus of any preceding embodiment, wherein the acquired motion and proximity data comprises data captured when the person moves into a bed.

EMBODIMENT 23. The apparatus of any preceding embodiment, wherein the acquired motion and proximity data comprises data captured when the person falls down.

EMBODIMENT 24. The apparatus of any preceding embodiment, wherein the apparatus is a first apparatus, the transmitter of the first apparatus is configured to transmit the one or more physiological and/or behavioral features of the person to a second apparatus.

EMBODIMENT 25. The apparatus of any preceding embodiment, wherein the apparatus is one of a plurality of apparatuses that forms a mesh network configured for sharing data.

EMBODIMENT 26. The apparatus of any preceding embodiment, further comprising a wearable sensor from a list of a pulse-oximeter, a heart rate monitor, an ultrasound sensor, or a thermometer, or a nearable sensor from a list of an accelerometer, a pressure sensor, optical sensor, including a video-, infrared- or laser-based sensor, a capacitive or touch sensor, or an environmental sensor.

EMBODIMENT 27. The apparatus of embodiment 26, wherein the apparatus is a hub configured collecting sensor data from one or more of the wearable sensor or nearable sensor and transmitting the collected sensor data to the remote device.

EMBODIMENT 28. The apparatus of any preceding embodiment, further comprising: a microphone and a speaker.

EMBODIMENT 29. The apparatus of embodiment 28, wherein the microphone and the speaker are configured for communicating with a health care professional or caretaker.

EMBODIMENT 30. The apparatus of embodiment 28, wherein the microphone is used for monitoring a physiological function of the person and the physiological function includes one of respiration, coughing, or snoring.

EMBODIMENT 31. The apparatus of embodiment 28, wherein the microphone is used for monitoring noise in an environment of the person.

EMBODIMENT 32. The apparatus of embodiment 28, wherein the microphone is used for monitoring a behavior of the person and the behavior includes one of TV watching, going to bed, or falling down.

EMBODIMENT 33. The apparatus of any preceding embodiment, further comprising: a light sensor configured for monitoring a light level of an environment of the person.

EMBODIMENT 34. The apparatus of embodiment 33, wherein the light sensor is used for monitoring a bed time of the person.

EMBODIMENT 35. The apparatus of embodiment 34, wherein the bed time is determined when the light sensor detects that a light in the environment of the person is turned off.

EMBODIMENT 36. The apparatus of any preceding embodiment, wherein the transmitter comprises a wired communication component configured to work over Ethernet or USB protocol.

EMBODIMENT 37. The apparatus of any preceding embodiment, wherein the transmitter comprises a wireless communication component configured to work over Bluetooth, Wi-Fi, a local area network, a wide area network, or a cellular network.

EMBODIMENT 38. The apparatus of any preceding embodiment, further comprising a user interface having one or more buttons for collecting one or more inputs.

EMBODIMENT 39. The apparatus of embodiment 38, wherein the one or more buttons are configured for activating or inactivating a system functionality, for communicating with a health care professional or caretaker, or for storing timestamps of events identifying a bed time, a rise time, or a bed exit.

EMBODIMENT 40. The apparatus of any preceding embodiment, further comprising one or more LED lights to provide a status indicator of the apparatus, wherein the status indicator indicates a status of power, connectivity, configuration, or a status of measured data from a list of sleep quality, respiration signal quality, or successful data collection.

EMBODIMENT 41. A method for non-contact monitoring of a person, the method comprising: acquiring, via a radar system, motion and proximity data of the person at a plurality of distances; storing, in a memory, the acquired motion and proximity data; processing, via a processor coupled to the memory, the acquired motion and proximity data; identifying, via the processor, one or more physiological and/or behavioral features of the person based on the processed motion and proximity data; and transmitting, via a transmitter, the one or more physiological and/or behavioral features of the person to a remote device.

EMBODIMENT 42. The method of any preceding embodiment, further comprising: transmitting the acquired motion and proximity data of the person to the remote device for data analysis and processing.

EMBODIMENT 43. The method of any preceding embodiment, wherein the radar system comprises a coherent pulsed ultra-wide band radar.

EMBODIMENT 44. The method of any preceding embodiment, wherein the radar system is a multistatic radar system configured for acquiring motion and proximity data of a plurality of persons.

EMBODIMENT 45. The method of any preceding embodiment, wherein the multistatic radar system is configured for beamforming to direct a radar beam to one or more specific anatomical portions of at least one person of the plurality of persons.

EMBODIMENT 46. The method of any preceding embodiment, further comprising: determining a position of the person based on the acquired motion and proximity data at the plurality of distances.

EMBODIMENT 47. The method of any preceding embodiment, wherein the radar system is configured for acquiring the motion and proximity data of the person at the plurality of distances between 0.01 m and 30 m from the radar system.

EMBODIMENT 48. The method of any preceding embodiment, wherein the radar system is configured for acquiring the motion and proximity data of the person at the plurality of distances between 0.3 m and 3.2 m from the radar system.

EMBODIMENT 49. The method of embodiment 47, further comprising: identifying, via the processor, the person from other people present between 0.01 m and 30 m from the radar system.

EMBODIMENT 50. The method of embodiment 48, further comprising: identifying, via the processor, the person from other people present between 0.3 m and 3.2 m from the radar system.

EMBODIMENT 51. The method of any preceding embodiment, wherein the acquired motion and proximity data comprises respiratory-induced body movements from thoracic and abdominal areas of the person.

EMBODIMENT 52. The method of any preceding embodiment, wherein the acquired motion and proximity data comprises vital signs of the person.

EMBODIMENT 53. The method of any preceding embodiment, wherein the acquired motion and proximity data is used to determine a breathing pattern of the person.

EMBODIMENT 54. The method of any preceding embodiment, wherein the acquired motion and proximity data is used to monitor a heart activity of the person.

EMBODIMENT 55. The method of any preceding embodiment, wherein the acquired motion and proximity data is used to monitor behavior of the person, and wherein the behavior comprises one of bed occupancy, activity, or sleep behavior.

EMBODIMENT 56. The method of any preceding embodiment, wherein the one or more behavioral features of the person comprises sleep behavior of the person.

EMBODIMENT 57. The method of embodiment 56, wherein the sleep behavior of the person comprises a pattern of sleep stages that the person goes through during sleep.

EMBODIMENT 58. The method of any preceding embodiment, wherein the acquired motion and proximity data comprises data captured while the person is asleep.

EMBODIMENT 59. The method of any preceding embodiment, wherein the acquired motion and proximity data comprises data captured while the person is awake in bed.

EMBODIMENT 60. The method of any preceding embodiment, wherein the acquired motion and proximity data comprises data captured while the person moves around in a vicinity of the radar system.

EMBODIMENT 61. The method of any preceding embodiment, wherein the acquired motion and proximity data comprises data captured when the person moves out of a bed.

EMBODIMENT 62. The method of any preceding embodiment, wherein the acquired motion and proximity data comprises data captured when the person moves into a bed.

EMBODIMENT 63. The method of any preceding embodiment, wherein the acquired motion and proximity data comprises data captured when the person falls down.

EMBODIMENT 64. The method of any preceding embodiment, further comprising: transmitting, via the transmitter, the one or more physiological and/or behavioral features of the person to one or more apparatuses in a mesh network.

EMBODIMENT 65. The method of embodiment 64, further comprising: sharing data with the one or more apparatuses in the mesh network.

EMBODIMENT 66. The method of any preceding embodiment, further comprising: collecting, via a wearable sensor, sensor data of the person, wherein the wearable sensor is one from a list of a pulse-oximeter, a heart rate monitor, an ultrasound sensor, or a thermometer, or from a nearable sensor from a list of an accelerometer, a pressure sensor, optical sensor, including a video-, infrared- or laser-based sensor, a capacitive or touch sensor, or an environmental sensor.

EMBODIMENT 67. The method of embodiment 66, further comprising: transmitting the collected sensor data to the remote device.

EMBODIMENT 68. The method of any preceding embodiment, further comprising: communicating, via a microphone and a speaker, with a health care professional or caretaker.

EMBODIMENT 69. The method of any preceding embodiment, further comprising:
monitoring, via a microphone, a physiological function of the person.

EMBODIMENT 70. The method of embodiment 69, wherein the physiological function includes one of respiration, coughing, or snoring.

EMBODIMENT 71. The method of any preceding embodiment, further comprising: monitoring, via a microphone, noise in an environment of the person.

EMBODIMENT 72. The method of any preceding embodiment, further comprising: monitoring, via a microphone, a behavior of the person, wherein the behavior includes one of TV watching, going to bed, or falling down.

EMBODIMENT 73. The method of any preceding embodiment, further comprising: monitoring, via a light sensor, a light level of an environment of the person.

EMBODIMENT 74. The method of any preceding embodiment, further comprising: determining, via a light sensor, a bed time of the person.

EMBODIMENT 75. The method of embodiment 74, wherein the bed time is determined when the light sensor detects that a light in an environment of the person is turned off.

EMBODIMENT 76. The method of any preceding embodiment, wherein the transmitter comprises a wired communication component configured to work over Ethernet or USB protocol.

EMBODIMENT 77. The method of any preceding embodiment, wherein the transmitter comprises a wireless communication component configured to work over Bluetooth, Wi-Fi, a local area network, a wide area network, or a cellular network.

EMBODIMENT 78. The method of any preceding embodiment, further comprising: collecting one or more inputs via a user interface having one or more buttons.

EMBODIMENT 79. The method of embodiment 78, further comprising: activating or inactivating, via the one or more buttons of the user interface, a system functionality; communicating with, via the one or more buttons of the user interface, a health care professional or caretaker; or storing, via the one or more buttons of the user interface, one or more timestamps of events identifying a bed time, a rise time, or a bed exit.

EMBODIMENT 80. The method of any preceding embodiment, further comprising: providing, via one or more LED lights, a status indicator to indicate a status of power, connectivity, configuration, or a status of measured data from a list of sleep quality, respiration signal quality, or successful data collection.

EMBODIMENT 81. A system for monitoring a plurality of patients, comprising: a plurality of apparatuses in a mesh network comprising at least a first apparatus and a second apparatus, wherein the first apparatus is positioned at a first location within a first local area, wherein the first apparatus comprises: a radar system configured for acquiring motion and proximity data of at least a first patient from the plurality of patients at a plurality of distances; a processor configured for processing the acquired motion and proximity data to identify one or more physiological and/or behavioral features of at least the first patient from the plurality of patients; and a transmitter configured for transmitting the one or more physiological and/or behavioral features of at least the first patient from the plurality of patients to the second apparatus in the mesh network or a remote server.

EMBODIMENT 82. The system of embodiment 81, wherein the transmitter is configured for transmitting the acquired motion and proximity data of at least the first patient from the plurality of patients to the second apparatus in the mesh network or the remote server.

EMBODIMENT 83. The system of any preceding embodiment, wherein the radar system comprises a coherent pulsed ultra-wide band radar.

EMBODIMENT 84. The system of any preceding embodiment, wherein the radar system is a multistatic radar system configured for acquiring motion and proximity data of at least the first patient from the plurality of patients.

EMBODIMENT 85. The system of embodiment 84, wherein the multistatic radar system is configured for beamforming to direct a radar beam to one or more specific anatomical portions of at least the first patient from the plurality of patients.

EMBODIMENT 86. The system of embodiment 84, wherein the multistatic radar system is configured for capturing a position of at least the first patient from the plurality of patients at the plurality of distances.

EMBODIMENT 87. The system of any preceding embodiment, wherein the radar system is configured for acquiring the motion and proximity data of at least the first patient from the plurality of patients at the plurality of distances between 0.01 m and 30 m from the first apparatus.

EMBODIMENT 88. The system of any preceding embodiment, wherein the radar system is configured for acquiring the motion and proximity data of at least the first patient from the plurality of patients at the plurality of distances between 0.3 m and 3.2 m from the first apparatus.

EMBODIMENT 89. The system of embodiment 87, wherein the acquired motion and proximity data of at least the first patient from the plurality of patients are processed by the processor for identifying at least the first patient from other patients present between 0.01 m and 30 m from the first apparatus.

EMBODIMENT 90. The system of embodiment 88, wherein the acquired motion and proximity data of at least the first patient from the plurality of patients are processed by the processor for identifying at least the first patient from other patients present between 0.3 m and 3.2 m from the first apparatus.

EMBODIMENT 91. The system of any preceding embodiment, wherein the acquired motion and proximity data comprises respiratory-induced body movements from thoracic and abdominal areas of at least the first patient from the plurality of patients.

EMBODIMENT 92. The system of any preceding embodiment, wherein the acquired motion and proximity data comprises vital signs of at least the first patient from the plurality of patients.

EMBODIMENT 93. The system of any preceding embodiment, wherein the acquired motion and proximity data is used to determine a breathing pattern of at least the first patient from the plurality of patients.

EMBODIMENT 94. The system of any preceding embodiment, wherein the acquired motion and proximity data is used to monitor a heart activity of at least the first patient from the plurality of patients.

EMBODIMENT 95. The system of any preceding embodiment, wherein the acquired motion and proximity data is used to monitor behavior of at least the first patient from the plurality of patients, and wherein the behavior comprises one of bed occupancy, activity, or sleep behavior.

EMBODIMENT 96. The system of any preceding embodiment, wherein the one or more behavioral features of at least the first patient from the plurality of patients comprises sleep behavior of at least the first patient from the plurality of patients.

EMBODIMENT 97. The system of embodiment 96, wherein the sleep behavior comprises a pattern of sleep stages that at least the first patient from the plurality of patients goes through during sleep.

EMBODIMENT 98. The system of any preceding embodiment, wherein the acquired motion and proximity data comprises data captured while at least the first patient from the plurality of patients is asleep.

EMBODIMENT 99. The system of any preceding embodiment, wherein the acquired motion and proximity data comprises data captured while at least the first patient from the plurality of patients is awake in bed.

EMBODIMENT 100. The system of any preceding embodiment, wherein the acquired motion and proximity data comprises data captured while at least the first patient from the plurality of patients moves around in a vicinity of the first apparatus.

EMBODIMENT 101. The system of any preceding embodiment, wherein the acquired motion and proximity data comprises data captured while at least the first patient from the plurality of patients moves out of a bed.

EMBODIMENT 102. The system of any preceding embodiment, wherein the acquired motion and proximity data comprises data captured while at least the first patient from the plurality of patients moves into a bed.

EMBODIMENT 103. The system of any preceding embodiment, wherein the acquired motion and proximity data comprises data captured when at least the first patient from the plurality of patients falls down.

EMBODIMENT 104. The system of any preceding embodiment, wherein the plurality of apparatuses in the mesh network are configured for sharing data within the mesh network and with the remote server.

EMBODIMENT 105. The system of any preceding embodiment, wherein the second apparatus comprises a second radar system and is positioned at a second location within a second local area, and wherein the second radar system is configured for acquiring motion and proximity data of a second patient.

EMBODIMENT 106. The system of embodiment 105, wherein the second apparatus further comprises a second processor configured for processing the acquired motion and proximity data of the second patient to identify one or more physiological and/or behavioral features of the second patient, and a second transmitter configured for transmitting the one or more physiological and/or behavioral features of the second patient to the first apparatus in the mesh network or the remote server.

EMBODIMENT 107. The system of any preceding embodiment, wherein the first apparatus further comprises a wearable sensor from a list of a pulse-oximeter, a heart rate monitor, an ultrasound sensor, or a thermometer, or a nearable sensor from a list of an accelerometer, a pressure sensor, optical sensor, including a video-, infrared- or laser-based sensor, a capacitive or touch sensor, or an environmental sensor.

EMBODIMENT 108. The system of embodiment 107, wherein the first apparatus is a hub configured collecting sensor data from one or more of the wearable sensor or nearable sensor and transmitting the collected sensor data to the second apparatus or the remote server.

EMBODIMENT 109. The system of any preceding embodiment, wherein the first apparatus further comprises a microphone and a speaker configured for communicating with a health care professional or caretaker.

EMBODIMENT 110. The system of embodiment 109, wherein the microphone is used for monitoring a physiological function of at least the first patient from the plurality of patients and the physiological function includes one of respiration, coughing, or snoring.

EMBODIMENT 111. The system of embodiment 109, wherein the microphone is used for monitoring noise in an environment of at least the first patient from the plurality of patients.

EMBODIMENT 112. The system of embodiment 109, wherein the microphone is used for monitoring a behavior of at least the first patient from the plurality of patients and the behavior includes one of TV watching, going to bed, or falling down.

EMBODIMENT 113. The system of any preceding embodiment, further comprising: a light sensor configured for monitoring a light level of an environment of at least the first patient from the plurality of patients.

EMBODIMENT 114. The system of embodiment 113, wherein the light sensor is used for monitoring a bed time of at least the first patient from the plurality of patients.

EMBODIMENT 115. The system of embodiment 114, wherein the bed time is determined when the light sensor detects that a light in the environment of at least the first patient from the plurality of patients is turned off.

EMBODIMENT 116. The system of any preceding embodiment, wherein the transmitter comprises a wired communication component configured to work over Ethernet or USB protocol.

EMBODIMENT 117. The system of any preceding embodiment, wherein the transmitter comprises a wireless communication component configured to work over Bluetooth, Wi-Fi, a local area network, a wide area network, or a cellular network.

EMBODIMENT 118. The system of any preceding embodiment, wherein the first apparatus further comprises a user interface having one or more buttons for collecting one or more inputs.

EMBODIMENT 119. The system of embodiment 118, wherein the one or more buttons are configured for activating or inactivating a functionality of the first apparatus, for communicating with a health care professional or caretaker, or for storing timestamps of events identifying a bed time, a rise time, or a bed exit.

EMBODIMENT 120. The system of any preceding embodiment, wherein the first apparatus further comprises one or more LED lights to provide a status indicator of the first apparatus, wherein the status indicator indicates a status of power, connectivity, configuration, or a status of measured data from a list of sleep quality, respiration signal quality, or successful data collection.

EMBODIMENT 121. A method for monitoring a plurality of patients, comprising: configuring a plurality of apparatuses in a mesh network comprising at least a first apparatus and a second apparatus, wherein the first apparatus is positioned at a first location within a first local area, wherein at least a first patient from the plurality of patients is present at the first location and being monitored by the first apparatus; acquiring, via a radar system of the first apparatus, motion and proximity data of at least the first patient from the plurality of patients at a plurality of distances; storing, in a memory of the first apparatus, the acquired motion and proximity data; processing, via a processor coupled to the memory, the acquired motion and proximity data; identifying, via the processor, one or more physiological and/or behavioral features of at least the first patient from the plurality of patients based on the processed motion and proximity data; and transmitting, via a transmitter of the first apparatus, the one or more physiological and/or behavioral features of at least the first patient from the plurality of patients to the second apparatus in the mesh network or a remote server.

EMBODIMENT 122. The method of embodiment 121, further comprising: transmitting the acquired motion and proximity data of at least the first patient from the plurality of patients to the second apparatus in the mesh network or the remote server.

EMBODIMENT 123. The method of any preceding embodiment, wherein the radar system comprises a coherent pulsed ultra-wide band radar.

EMBODIMENT 124. The method of any preceding embodiment, wherein the radar system is a multistatic radar system configured for acquiring motion and proximity data of at least the first patient from the plurality of patients.

EMBODIMENT 125. The method of embodiment 124, wherein the multistatic radar system is configured for beamforming to direct a radar beam to one or more specific anatomical portions of at least the first patient from the plurality of patients.

EMBODIMENT 126. The method of any preceding embodiment, wherein the radar system is configured for acquiring the motion and proximity data of at least the first patient from the plurality of patients at the plurality of distances between 0.01 m and 30 m from the first apparatus.

EMBODIMENT 127. The method of any preceding embodiment, wherein the radar system is configured for acquiring the motion and proximity data of at least the first patient from the plurality of patients at the plurality of distances between 0.3 m and 3.2 m from the first apparatus.

EMBODIMENT 128. The method of embodiment 126, further comprising: identifying, via the processor, at least the first patient from other patients present between 0.01 m and 30 m from the first apparatus.

EMBODIMENT 129. The method of embodiment 127, further comprising: identifying, via the processor, at least the first patient from other patients present between 0.3 m and 3.2 m from the first apparatus.

EMBODIMENT 130. The method of any preceding embodiment, wherein the acquired motion and proximity data comprises respiratory-induced body movements from thoracic and abdominal areas of at least the first patient from the plurality of patients.

EMBODIMENT 131. The method of any preceding embodiment, wherein the acquired motion and proximity data comprises vital signs of at least the first patient from the plurality of patients.

EMBODIMENT 132. The method of any preceding embodiment, wherein the acquired motion and proximity data is used to determine a breathing pattern of at least the first patient from the plurality of patients.

EMBODIMENT 133. The method of any preceding embodiment, wherein the acquired motion and proximity data is used to monitor a heart activity of at least the first patient from the plurality of patients.

EMBODIMENT 134. The method of any preceding embodiment, wherein the acquired motion and proximity data is used to monitor behavior of at least the first patient from the plurality of patients, and wherein the behavior comprises one of bed occupancy, activity, or sleep behavior.

EMBODIMENT 135. The method of any preceding embodiment, wherein the one or more behavioral features of at least the first patient from the plurality of patients comprises sleep behavior of at least the first patient from the plurality of patients.

EMBODIMENT 136. The method of embodiment 135, wherein the sleep behavior comprises a pattern of sleep stages that at least the first patient from the plurality of patients goes through during sleep.

EMBODIMENT 137. The method of any preceding embodiment, wherein the acquired motion and proximity data comprises data captured while at least the first patient from the plurality of patients is asleep.

EMBODIMENT 138. The method of any preceding embodiment, wherein the acquired motion and proximity data comprises data captured while at least the first patient from the plurality of patients is awake in bed.

EMBODIMENT 139. The method of any preceding embodiment, wherein the acquired motion and proximity data comprises data captured while at least the first patient from the plurality of patients moves around in a vicinity of the first apparatus.

EMBODIMENT 140. The method of any preceding embodiment, wherein the acquired motion and proximity data comprises data captured while at least the first patient from the plurality of patients moves out of a bed.

EMBODIMENT 141. The method of any preceding embodiment, wherein the acquired motion and proximity data comprises data captured while at least the first patient from the plurality of patients moves into a bed.

EMBODIMENT 142. The method of any preceding embodiment, wherein the acquired motion and proximity data comprises data captured when at least the first patient from the plurality of patients falls down.

EMBODIMENT 143. The method of any preceding embodiment, wherein the plurality of apparatuses in the mesh network are configured for sharing data within the mesh network and with the remote server.

EMBODIMENT 144. The method of any preceding embodiment, wherein the second apparatus comprises a second radar system and is positioned at a second location within a second local area, the method further comprising: acquiring, via the second radar system, motion and proximity data of a second patient.

EMBODIMENT 145. The method of embodiment 144, wherein the second apparatus further comprises a second processor and a second transmitter, the method further comprising: processing, via the second processor, the acquired motion and proximity data of the second patient; identifying, via the second processor, one or more physiological and/or behavioral features of the second patient based on the processed motion and proximity data of the second patient; and transmitting, via the second transmitter, the one or more physiological and/or behavioral features of the second patient to the first apparatus in the mesh network or the remote server.

EMBODIMENT 146. The method of any preceding embodiment, further comprising: collecting, via a wearable sensor, sensor data of at least the first patient from the plurality of patients, wherein the wearable sensor is one from a list of a pulse-oximeter, a heart rate monitor, an ultrasound sensor, or a thermometer, or from a nearable sensor from a list of an accelerometer, a pressure sensor, optical sensor, including a video-, infrared- or laser-based sensor, a capacitive or touch sensor, or an environmental sensor.

EMBODIMENT 147. The method of embodiment 146, further comprising: transmitting the collected sensor data to the second apparatus or the remote server.

EMBODIMENT 148. The method of any preceding embodiment, further comprising: communicating, via a microphone and a speaker, with a health care professional or caretaker.

EMBODIMENT 149. The method of any preceding embodiment, further comprising: monitoring, via a microphone, a physiological function of at least the first patient from the plurality of patients.

EMBODIMENT 150. The method of embodiment 149, wherein the physiological function includes one of respiration, coughing, or snoring.

EMBODIMENT 151. The method of any preceding embodiment, further comprising: monitoring, via a microphone, noise in an environment of at least the first patient from the plurality of patients.

EMBODIMENT 152. The method of any preceding embodiment, further comprising: monitoring, via a microphone, a behavior of at least the first patient from the plurality of patients, wherein the behavior includes one of TV watching, going to bed, or falling down.

EMBODIMENT 153. The method of any preceding embodiment, further comprising: monitoring, via a light sensor, a light level of an environment of at least the first patient from the plurality of patients.

EMBODIMENT 154. The method of any preceding embodiment, further comprising: monitoring, via a light sensor, a bed time of at least the first patient from the plurality of patients.

EMBODIMENT 155. The method of embodiment 154, wherein the bed time is determined when the light sensor detects that a light in an environment of at least the first patient from the plurality of patients is turned off.

EMBODIMENT 156. The method of any preceding embodiment, wherein the transmitting comprises transmitting via a wired communication component over Ethernet or USB protocol.

EMBODIMENT 157. The method of any preceding embodiment, wherein the transmitting comprises transmitting via a wireless communication component over Bluetooth, Wi-Fi, a local area network, a wide area network, or a cellular network.

EMBODIMENT 158. The method of any preceding embodiment, further comprising: collecting one or more inputs via a user interface of the first apparatus, the user interface having one or more buttons.

EMBODIMENT 159. The method of embodiment 158, further comprising: activating or inactivating, via the one or more buttons of the user interface, an apparatus functionality; communicating with, via the one or more buttons of the user interface, a health care professional or caretaker; or storing, via the one or more buttons of the user interface, one or more timestamps of events identifying a bed time, a rise time, or a bed exit.

EMBODIMENT 160. The method of any preceding embodiment, further comprising: providing, via one or more LED lights of the first apparatus, a status indicator of the first apparatus to indicate a status of power, connectivity, configuration, or a status of measured data from a list of sleep quality, respiration signal quality, or successful data collection.

As will be readily appreciated by those having ordinary skill in the art after becoming familiar with the teachings herein, a number of variations are possible on the examples and embodiments described above. The logical operations making up the embodiments of the technology described herein are referred to variously as operations, steps, objects, elements, components, or modules. Furthermore, it should be understood that these may be performed in any order, unless explicitly claimed otherwise or a specific order is inherently necessitated by the description.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the monitoring system. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary embodiments of the vital sign monitoring system as defined herein. Although various embodiments of the subject matter have been described above with a certain degree of particularity, or with reference to one or more individual embodiments, those skilled in the art could make numerous alterations to the disclosed embodiments without departing from the spirit or scope of the described subject matter.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined herein.

What is claimed is:

1. An apparatus for non-contact monitoring of a person, comprising:
a radar system configured for acquiring motion and proximity data of a person at a plurality of distances;
a processor configured for processing the acquired motion and proximity data to identify one or more physiological and/or behavioral features of the person; and
a transmitter configured for transmitting the one or more physiological and/or behavioral features of the person to a remote device.

2. The apparatus of claim 1, wherein the transmitter is configured for transmitting the acquired motion and proximity data of the person to the remote device.

3. The apparatus of claim 1, wherein the radar system comprises a coherent pulsed ultra-wide band radar.

4. The apparatus of claim 1, wherein the radar system is a multistatic radar system configured for acquiring motion and proximity data of a plurality of persons.

5. The apparatus of claim 4, wherein the multistatic radar system is configured for beamforming to direct a radar beam to one or more specific anatomical portions of at least one person of the plurality of persons.

6. The apparatus of claim 1, wherein the radar system is configured for determining a position of the person based on the acquired motion and proximity data at the plurality of distances.

7. The apparatus of claim 1, wherein the radar system is configured for acquiring the motion and proximity data of the person at the plurality of distances between 0.01 m and 30 m from the apparatus.

8. The apparatus of claim 1, wherein the radar system is configured for acquiring the motion and proximity data of the person at the plurality of distances between 0.3 m and 3.2 m from the apparatus.

9. The apparatus of claim 7, wherein the acquired motion and proximity data of the person are processed by the processor for identifying the person from other people present between 0.01 m and 30 m from the apparatus.

10. The apparatus of claim 8, wherein the acquired motion and proximity data of the person are processed by the processor for identifying the person from other people present within the distance between 0.3 m and 3.2 m from the apparatus.

11. The apparatus of claim 1, wherein the acquired motion and proximity data comprises respiratory-induced body movements from thoracic and abdominal areas of the person.

12. The apparatus of claim 1, wherein the acquired motion and proximity data comprises vital signs of the person.

13. The apparatus of claim 1, wherein the acquired motion and proximity data is used to determine a breathing pattern of the person.

14. The apparatus of claim 1, wherein the acquired motion and proximity data is used to monitor a heart activity of the person.

15. The apparatus of claim 1, wherein the acquired motion and proximity data is used to monitor behavior of the person, and wherein the behavior comprises one of bed occupancy, activity, or sleep behavior.

16. The apparatus of claim 1, wherein the one or more behavioral features of the person comprises sleep behavior of the person.

17. The apparatus of claim 16, wherein the sleep behavior comprises a pattern of sleep stages that the person goes through during sleep.

18. The apparatus of claim 1, wherein the acquired motion and proximity data comprises data captured while the person is asleep.

19. The apparatus of claim 1, wherein the acquired motion and proximity data comprises data captured while the person is awake in bed.

20. The apparatus of claim 1, wherein the acquired motion and proximity data comprises data captured while the person moves around in a vicinity of the apparatus.

21. The apparatus of claim 1, wherein the acquired motion and proximity data comprises data captured when the person moves out of a bed.

22. The apparatus of claim 1, wherein the acquired motion and proximity data comprises data captured when the person moves into a bed.

23. The apparatus of claim 1, wherein the acquired motion and proximity data comprises data captured when the person falls down.

24. The apparatus of claim 1, wherein the apparatus is a first apparatus, the transmitter of the first apparatus is configured to transmit the one or more physiological and/or behavioral features of the person to a second apparatus.

25. The apparatus of claim 1, wherein the apparatus is one of a plurality of apparatuses that forms a mesh network configured for sharing data.

26. The apparatus of claim 1, further comprising a wearable sensor from a list of a pulse-oximeter, a heart rate monitor, an ultrasound sensor, or a thermometer, or a nearable sensor from a list of an accelerometer, a pressure sensor, optical sensor, including a video-, infrared- or laser-based sensor, a capacitive or touch sensor, or an environmental sensor.

27. The apparatus of claim 26, wherein the apparatus is a hub configured collecting sensor data from one or more of the wearable sensor or nearable sensor and transmitting the collected sensor data to the remote device.

28. The apparatus of claim 1, further comprising: a microphone and a speaker.

29. The apparatus of claim 28, wherein the microphone and the speaker are configured for communicating with a health care professional or caretaker.

30. The apparatus of claim 28, wherein the microphone is used for monitoring a physiological function of the person and the physiological function includes one of respiration, coughing, or snoring.

31. The apparatus of claim 28, wherein the microphone is used for monitoring noise in an environment of the person.

32. The apparatus of claim 28, wherein the microphone is used for monitoring a behavior of the person and the behavior includes one of TV watching, going to bed, or falling down.

33. The apparatus of claim 1, further comprising: a light sensor configured for monitoring a light level of an environment of the person.

34. The apparatus of claim 33, wherein the light sensor is used for monitoring a bed time of the person.

35. The apparatus of claim 34, wherein the bed time is determined when the light sensor detects that a light in the environment of the person is turned off.

36. The apparatus of claim 1, wherein the transmitter comprises a wired communication component configured to work over Ethernet or USB protocol.

37. The apparatus of claim 1, wherein the transmitter comprises a wireless communication component configured to work over Bluetooth, Wi-Fi, a local area network, a wide area network, or a cellular network.

38. The apparatus of claim 1, further comprising a user interface having one or more buttons for collecting one or more inputs.

39. The apparatus of claim 38, wherein the one or more buttons are configured for activating or inactivating a system functionality, for communicating with a health care professional or caretaker, or for storing timestamps of events identifying a bed time, a rise time, or a bed exit.

40. The apparatus of claim 1, further comprising one or more LED lights to provide a status indicator of the apparatus, wherein the status indicator indicates a status of power, connectivity, configuration, or a status of measured data from a list of sleep quality, respiration signal quality, or successful data collection.

41. A method for non-contact monitoring of a person, the method comprising:
acquiring, via a radar system, motion and proximity data of the person at a plurality of distances;
storing, in a memory, the acquired motion and proximity data;
processing, via a processor coupled to the memory, the acquired motion and proximity data;
identifying, via the processor, one or more physiological and/or behavioral features of the person based on the processed motion and proximity data; and
transmitting, via a transmitter, the one or more physiological and/or behavioral features of the person to a remote device.

42. The method of claim 41, further comprising:
transmitting the acquired motion and proximity data of the person to the remote device for data analysis and processing.

43. The method of claim 41, wherein the radar system comprises a coherent pulsed ultra-wide band radar.

44. The method of claim 41, wherein the radar system is a multistatic radar system configured for acquiring motion and proximity data of a plurality of persons.

45. The method of claim 41, wherein the multistatic radar system is configured for beamforming to direct a radar beam to one or more specific anatomical portions of at least one person of the plurality of persons.

46. The method of claim 41, further comprising:
determining a position of the person based on the acquired motion and proximity data at the plurality of distances.

47. The method of claim 41, wherein the radar system is configured for acquiring the motion and proximity data of the person at the plurality of distances between 0.01 m and 30 m from the radar system.

48. The method of claim 41, wherein the radar system is configured for acquiring the motion and proximity data of the person at the plurality of distances between 0.3 m and 3.2 m from the radar system.

49. The method of claim 47, further comprising:
identifying, via the processor, the person from other people present between 0.01 m and 30 m from the radar system.

50. The method of claim 48, further comprising:
identifying, via the processor, the person from other people present between 0.3 m and 3.2 m from the radar system.

51. The method of claim 41, wherein the acquired motion and proximity data comprises respiratory-induced body movements from thoracic and abdominal areas of the person.

52. The method of claim 41, wherein the acquired motion and proximity data comprises vital signs of the person.

53. The method of claim 41, wherein the acquired motion and proximity data is used to determine a breathing pattern of the person.

54. The method of claim 41, wherein the acquired motion and proximity data is used to monitor a heart activity of the person.

55. The method of claim 41, wherein the acquired motion and proximity data is used to monitor behavior of the person, and wherein the behavior comprises one of bed occupancy, activity, or sleep behavior.

56. The method of claim 41, wherein the one or more behavioral features of the person comprises sleep behavior of the person.

57. The method of claim 56, wherein the sleep behavior of the person comprises a pattern of sleep stages that the person goes through during sleep.

58. The method of claim 41, wherein the acquired motion and proximity data comprises data captured while the person is asleep.

59. The method of claim 41, wherein the acquired motion and proximity data comprises data captured while the person is awake in bed.

60. The method of claim 41, wherein the acquired motion and proximity data comprises data captured while the person moves around in a vicinity of the radar system.

61. The method of claim 41, wherein the acquired motion and proximity data comprises data captured when the person moves out of a bed.

62. The method of claim 41, wherein the acquired motion and proximity data comprises data captured when the person moves into a bed.

63. The method of claim 41, wherein the acquired motion and proximity data comprises data captured when the person falls down.

64. The method of claim 41, further comprising:
transmitting, via the transmitter, the one or more physiological and/or behavioral features of the person to one or more apparatuses in a mesh network.

65. The method of claim 64, further comprising:
sharing data with the one or more apparatuses in the mesh network.

66. The method of claim 41, further comprising:
collecting, via a wearable sensor, sensor data of the person, wherein the wearable sensor is one from a list of a pulse-oximeter, a heart rate monitor, an ultrasound sensor, or a thermometer, or from a nearable sensor from a list of an accelerometer, a pressure sensor, optical sensor, including a video-, infrared- or laser-based sensor, a capacitive or touch sensor, or an environmental sensor.

67. The method of claim 66, further comprising:
transmitting the collected sensor data to the remote device.

68. The method of claim 41, further comprising:
communicating, via a microphone and a speaker, with a health care professional or caretaker.

69. The method of claim 41, further comprising:
monitoring, via a microphone, a physiological function of the person.

70. The method of claim 69, wherein the physiological function includes one of respiration, coughing, or snoring.

71. The method of claim 41, further comprising:
monitoring, via a microphone, noise in an environment of the person.

72. The method of claim 41, further comprising:
monitoring, via a microphone, a behavior of the person, wherein the behavior includes one of TV watching, going to bed, or falling down.

73. The method of claim 41, further comprising:
monitoring, via a light sensor, a light level of an environment of the person.

74. The method of claim 41, further comprising:
determining, via a light sensor, a bed time of the person.

75. The method of claim 74, wherein the bed time is determined when the light sensor detects that a light in an environment of the person is turned off.

76. The method of claim 41, wherein the transmitter comprises a wired communication component configured to work over Ethernet or USB protocol.

77. The method of claim 41, wherein the transmitter comprises a wireless communication component configured to work over Bluetooth, Wi-Fi, a local area network, a wide area network, or a cellular network.

78. The method of claim 41, further comprising:
collecting one or more inputs via a user interface having one or more buttons.

79. The method of claim 78, further comprising:
activating or inactivating, via the one or more buttons of the user interface, a system functionality;
communicating with, via the one or more buttons of the user interface, a health care professional or caretaker; or
storing, via the one or more buttons of the user interface, one or more timestamps of events identifying a bed time, a rise time, or a bed exit.

80. The method of claim 41, further comprising:
providing, via one or more LED lights, a status indicator to indicate a status of power, connectivity, configuration, or a status of measured data from a list of sleep quality, respiration signal quality, or successful data collection.

81. A system for monitoring a plurality of patients, comprising:
a plurality of apparatuses in a mesh network comprising at least a first apparatus and a second apparatus, wherein the first apparatus is positioned at a first location within a first local area, wherein the first apparatus comprises:
a radar system configured for acquiring motion and proximity data of at least a first patient from the plurality of patients at a plurality of distances;
a processor configured for processing the acquired motion and proximity data to identify one or more physiological and/or behavioral features of at least the first patient from the plurality of patients; and
a transmitter configured for transmitting the one or more physiological and/or behavioral features of at least the first patient from the plurality of patients to the second apparatus in the mesh network or a remote server.

82. The system of claim 81, wherein the transmitter is configured for transmitting the acquired motion and proximity data of at least the first patient from the plurality of patients to the second apparatus in the mesh network or the remote server.

83. The system of claim 81, wherein the radar system comprises a coherent pulsed ultra-wide band radar.

84. The system of claim 81, wherein the radar system is a multistatic radar system configured for acquiring motion and proximity data of at least the first patient from the plurality of patients.

85. The system of claim 84, wherein the multistatic radar system is configured for beamforming to direct a radar beam to one or more specific anatomical portions of at least the first patient from the plurality of patients.

86. The system of claim 84, wherein the multistatic radar system is configured for capturing a position of at least the first patient from the plurality of patients at the plurality of distances.

87. The system of claim 81, wherein the radar system is configured for acquiring the motion and proximity data of at least the first patient from the plurality of patients at the plurality of distances between 0.01 m and 30 m from the first apparatus.

88. The system of claim 81, wherein the radar system is configured for acquiring the motion and proximity data of at least the first patient from the plurality of patients at the plurality of distances between 0.3 m and 3.2 m from the first apparatus.

89. The system of claim 87, wherein the acquired motion and proximity data of at least the first patient from the plurality of patients are processed by the processor for identifying at least the first patient from other patients present between 0.01 m and 30 m from the first apparatus.

90. The system of claim 88, wherein the acquired motion and proximity data of at least the first patient from the plurality of patients are processed by the processor for identifying at least the first patient from other patients present between 0.3 m and 3.2 m from the first apparatus.

91. The system of claim 81, wherein the acquired motion and proximity data comprises respiratory-induced body movements from thoracic and abdominal areas of at least the first patient from the plurality of patients.

92. The system of claim 81, wherein the acquired motion and proximity data comprises vital signs of at least the first patient from the plurality of patients.

93. The system of claim 81, wherein the acquired motion and proximity data is used to determine a breathing pattern of at least the first patient from the plurality of patients.

94. The system of claim 81, wherein the acquired motion and proximity data is used to monitor a heart activity of at least the first patient from the plurality of patients.

95. The system of claim 81, wherein the acquired motion and proximity data is used to monitor behavior of at least the first patient from the plurality of patients, and wherein the behavior comprises one of bed occupancy, activity, or sleep behavior.

96. The system of claim 81, wherein the one or more behavioral features of at least the first patient from the plurality of patients comprises sleep behavior of at least the first patient from the plurality of patients.

97. The system of claim 96, wherein the sleep behavior comprises a pattern of sleep stages that at least the first patient from the plurality of patients goes through during sleep.

98. The system of claim 81, wherein the acquired motion and proximity data comprises data captured while at least the first patient from the plurality of patients is asleep.

99. The system of claim 81, wherein the acquired motion and proximity data comprises data captured while at least the first patient from the plurality of patients is awake in bed.

100. The system of claim 81, wherein the acquired motion and proximity data comprises data captured while at least the first patient from the plurality of patients moves around in a vicinity of the first apparatus.

101. The system of claim 81, wherein the acquired motion and proximity data comprises data captured while at least the first patient from the plurality of patients moves out of a bed.

102. The system of claim 81, wherein the acquired motion and proximity data comprises data captured while at least the first patient from the plurality of patients moves into a bed.

103. The system of claim 81, wherein the acquired motion and proximity data comprises data captured when at least the first patient from the plurality of patients falls down.

104. The system of claim 81, wherein the plurality of apparatuses in the mesh network are configured for sharing data within the mesh network and with the remote server.

105. The system of claim 81, wherein the second apparatus comprises a second radar system and is positioned at a second location within a second local area, and wherein the second radar system is configured for acquiring motion and proximity data of a second patient.

106. The system of claim 105, wherein the second apparatus further comprises a second processor configured for processing the acquired motion and proximity data of the second patient to identify one or more physiological and/or behavioral features of the second patient, and a second transmitter configured for transmitting the one or more physiological and/or behavioral features of the second patient to the first apparatus in the mesh network or the remote server.

107. The system of claim 81, wherein the first apparatus further comprises a wearable sensor from a list of a pulse-oximeter, a heart rate monitor, an ultrasound sensor, or a thermometer, or a nearable sensor from a list of an accelerometer, a pressure sensor, optical sensor, including a video-, infrared- or laser-based sensor, a capacitive or touch sensor, or an environmental sensor.

108. The system of claim 107, wherein the first apparatus is a hub configured collecting sensor data from one or more of the wearable sensor or nearable sensor and transmitting the collected sensor data to the second apparatus or the remote server.

109. The system of claim 81, wherein the first apparatus further comprises a microphone and a speaker configured for communicating with a health care professional or caretaker.

110. The system of claim 109, wherein the microphone is used for monitoring a physiological function of at least the first patient from the plurality of patients and the physiological function includes one of respiration, coughing, or snoring.

111. The system of claim 109, wherein the microphone is used for monitoring noise in an environment of at least the first patient from the plurality of patients.

112. The system of claim 109, wherein the microphone is used for monitoring a behavior of at least the first patient from the plurality of patients and the behavior includes one of TV watching, going to bed, or falling down.

113. The system of claim 81, further comprising: a light sensor configured for monitoring a light level of an environment of at least the first patient from the plurality of patients.

114. The system of claim 113, wherein the light sensor is used for monitoring a bed time of at least the first patient from the plurality of patients.

115. The system of claim 114, wherein the bed time is determined when the light sensor detects that a light in the environment of at least the first patient from the plurality of patients is turned off.

116. The system of claim 81, wherein the transmitter comprises a wired communication component configured to work over Ethernet or USB protocol.

117. The system of claim 81, wherein the transmitter comprises a wireless communication component configured to work over Bluetooth, Wi-Fi, a local area network, a wide area network, or a cellular network.

118. The system of claim 81, wherein the first apparatus further comprises a user interface having one or more buttons for collecting one or more inputs.

119. The system of claim 118, wherein the one or more buttons are configured for activating or inactivating a functionality of the first apparatus, for communicating with a health care professional or caretaker, or for storing timestamps of events identifying a bed time, a rise time, or a bed exit.

120. The system of claim 81, wherein the first apparatus further comprises one or more LED lights to provide a status indicator of the first apparatus, wherein the status indicator indicates a status of power, connectivity, configuration, or a status of measured data from a list of sleep quality, respiration signal quality, or successful data collection.

121. A method for monitoring a plurality of patients, comprising:
configuring a plurality of apparatuses in a mesh network comprising at least a first apparatus and a second apparatus, wherein the first apparatus is positioned at a first location within a first local area, wherein at least a first patient from the plurality of patients is present at the first location and being monitored by the first apparatus;
acquiring, via a radar system of the first apparatus, motion and proximity data of at least the first patient from the plurality of patients at a plurality of distances;
storing, in a memory of the first apparatus, the acquired motion and proximity data;
processing, via a processor coupled to the memory, the acquired motion and proximity data;
identifying, via the processor, one or more physiological and/or behavioral features of at least the first patient from the plurality of patients based on the processed motion and proximity data; and
transmitting, via a transmitter of the first apparatus, the one or more physiological and/or behavioral features of at least the first patient from the plurality of patients to the second apparatus in the mesh network or a remote server.

122. The method of claim 121, further comprising:
transmitting the acquired motion and proximity data of at least the first patient from the plurality of patients to the second apparatus in the mesh network or the remote server.

123. The method of claim 121, wherein the radar system comprises a coherent pulsed ultra-wide band radar.

124. The method of claim 121, wherein the radar system is a multistatic radar system configured for acquiring motion and proximity data of at least the first patient from the plurality of patients.

125. The method of claim 124, wherein the multistatic radar system is configured for beamforming to direct a radar beam to one or more specific anatomical portions of at least the first patient from the plurality of patients.

126. The method of claim 121, wherein the radar system is configured for acquiring the motion and proximity data of at least the first patient from the plurality of patients at the plurality of distances between 0.01 m and 30 m from the first apparatus.

127. The method of claim 121, wherein the radar system is configured for acquiring the motion and proximity data of at least the first patient from the plurality of patients at the plurality of distances between 0.3 m and 3.2 m from the first apparatus.

128. The method of claim 126, further comprising:
identifying, via the processor, at least the first patient from other patients present between 0.01 m and 30 m from the first apparatus.

129. The method of claim 127, further comprising:
identifying, via the processor, at least the first patient from other patients present between 0.3 m and 3.2 m from the first apparatus.

130. The method of claim 121, wherein the acquired motion and proximity data comprises respiratory-induced body movements from thoracic and abdominal areas of at least the first patient from the plurality of patients.

131. The method of claim 121, wherein the acquired motion and proximity data comprises vital signs of at least the first patient from the plurality of patients.

132. The method of claim 121, wherein the acquired motion and proximity data is used to determine a breathing pattern of at least the first patient from the plurality of patients.

133. The method of claim 121, wherein the acquired motion and proximity data is used to monitor a heart activity of at least the first patient from the plurality of patients.

134. The method of claim 121, wherein the acquired motion and proximity data is used to monitor behavior of at least the first patient from the plurality of patients, and wherein the behavior comprises one of bed occupancy, activity, or sleep behavior.

135. The method of claim 121, wherein the one or more behavioral features of at least the first patient from the plurality of patients comprises sleep behavior of at least the first patient from the plurality of patients.

136. The method of claim 135, wherein the sleep behavior comprises a pattern of sleep stages that at least the first patient from the plurality of patients goes through during sleep.

137. The method of claim 121, wherein the acquired motion and proximity data comprises data captured while at least the first patient from the plurality of patients is asleep.

138. The method of claim 121, wherein the acquired motion and proximity data comprises data captured while at least the first patient from the plurality of patients is awake in bed.

139. The method of claim 121, wherein the acquired motion and proximity data comprises data captured while at least the first patient from the plurality of patients moves around in a vicinity of the first apparatus.

140. The method of claim 121, wherein the acquired motion and proximity data comprises data captured while at least the first patient from the plurality of patients moves out of a bed.

141. The method of claim 121, wherein the acquired motion and proximity data comprises data captured while at least the first patient from the plurality of patients moves into a bed.

142. The method of claim 121, wherein the acquired motion and proximity data comprises data captured when at least the first patient from the plurality of patients falls down.

143. The method of claim 121, wherein the plurality of apparatuses in the mesh network are configured for sharing data within the mesh network and with the remote server.

144. The method of claim 121, wherein the second apparatus comprises a second radar system and is positioned at a second location within a second local area, the method further comprising:
acquiring, via the second radar system, motion and proximity data of a second patient.

145. The method of claim 144, wherein the second apparatus further comprises a second processor and a second transmitter, the method further comprising:
processing, via the second processor, the acquired motion and proximity data of the second patient;
identifying, via the second processor, one or more physiological and/or behavioral features of the second patient based on the processed motion and proximity data of the second patient; and
transmitting, via the second transmitter, the one or more physiological and/or behavioral features of the second patient to the first apparatus in the mesh network or the remote server.

146. The method of claim 121, further comprising:
collecting, via a wearable sensor, sensor data of at least the first patient from the plurality of patients, wherein the wearable sensor is one from a list of a pulse-oximeter, a heart rate monitor, an ultrasound sensor, or a thermometer, or from a nearable sensor from a list of an accelerometer, a pressure sensor, optical sensor, including a video-, infrared- or laser-based sensor, a capacitive or touch sensor, or an environmental sensor.

147. The method of claim 146, further comprising:
transmitting the collected sensor data to the second apparatus or the remote server.

148. The method of claim 121, further comprising:
communicating, via a microphone and a speaker, with a health care professional or caretaker.

149. The method of claim 121, further comprising:
monitoring, via a microphone, a physiological function of at least the first patient from the plurality of patients.

150. The method of claim 149, wherein the physiological function includes one of respiration, coughing, or snoring.

151. The method of claim 121, further comprising:
monitoring, via a microphone, noise in an environment of at least the first patient from the plurality of patients.

152. The method of claim 121, further comprising:
monitoring, via a microphone, a behavior of at least the first patient from the plurality of patients, wherein the behavior includes one of TV watching, going to bed, or falling down.

153. The method of claim 121, further comprising:
monitoring, via a light sensor, a light level of an environment of at least the first patient from the plurality of patients.

154. The method of claim 121, further comprising:
monitoring, via a light sensor, a bed time of at least the first patient from the plurality of patients.

155. The method of claim 154, wherein the bed time is determined when the light sensor detects that a light in an environment of at least the first patient from the plurality of patients is turned off.

156. The method of claim 121, wherein the transmitting comprises transmitting via a wired communication component over Ethernet or USB protocol.

157. The method of claim 121, wherein the transmitting comprises transmitting via a wireless communication component over Bluetooth, Wi-Fi, a local area network, a wide area network, or a cellular network.

158. The method of claim 121, further comprising:
collecting one or more inputs via a user interface of the first apparatus, the user interface having one or more buttons.

159. The method of claim 158, further comprising:
activating or inactivating, via the one or more buttons of the user interface, an apparatus functionality;
communicating with, via the one or more buttons of the user interface, a health care professional or caretaker; or
storing, via the one or more buttons of the user interface, one or more timestamps of events identifying a bed time, a rise time, or a bed exit.

160. The method of claim 121, further comprising:
providing, via one or more LED lights of the first apparatus, a status indicator of the first apparatus to indicate a status of power, connectivity, configuration, or a status of measured data from a list of sleep quality, respiration signal quality, or successful data collection.
